(19) United States
(12) Patent Application Publication
(10) Pub. No.: US 2024/0091366 A1
(43) Pub. Date: Mar. 21, 2024

(54) NOVEL ACID-SENSITIVE APTAMER TRIPTOLIDE CONJUGATE AND APPLICATIONS

(71) Applicant: CHENGDU UNIVERSITY OF TRADITIONAL CHINESE MEDICINE, Chengdu (CN)

(72) Inventors: Jun LU, Chengdu (CN); Yun DENG, Chengdu (CN); Yao CHEN, Chengdu (CN); Jirui YANG, Chengdu (CN); Yi ZUO, Chengdu (CN); Xiao LI, Chengdu (CN); Qing REN, Chengdu (CN)

(21) Appl. No.: 18/467,621

(22) Filed: Sep. 14, 2023

(30) Foreign Application Priority Data
Feb. 22, 2022 (CN) 202210163765.1

(51) Int. Cl. A61K 47/54; A61P 35/00

(52) U.S. Cl. CPC A61K 47/549; A61P 35/00

(57) ABSTRACT

The present invention relates to the field of pharmaceutical and chemical engineering, and specifically relates to a weakly acidic microenvironment-sensitive aptamer for tumors, a triptolide conjugate. The conjugate is formed by conjugation between the 14-position hydroxyl group of triptolide and the aptamer via an acetal ester linking bond, which is an acid-sensitive linking bond with a cleavage condition of (pH=3.5-6.5), which is much less pH-sensitive and is more likely to cleave under the tumor microenvironment. Based on the characteristics of the aptamer targeting the highly expressed proteins on the membrane surface of tumor cells, the conjugate delivered triptolide targeted to tumor cells and mediated endocytosis to reach the lysosome; based on the characteristics of the acidic environment of lysosomes, the acetal ester linking bond released intact triptolide in the lysosomal acidic environment, targeting and killing of tumor cells. It overcomes the defects of the prior art that the linking bond in the aptamer triptolide conjugate has low acid sensitivity and is not easy to cleave under the environment of cancer cells in vivo, resulting in insufficient targeted release of triptolide, which in turn leads to the ineffective inhibition of cancer cells.

### CROSS REFERENCE TO RELATED APPLICATION(S)

This application is a national phase application of International Appl. No. PCT/CN2023/074352, filed Feb. 3, 2023, and claims the benefit of Chinese Patent Application No. 202210163765.1, filed Feb. 22, 2022.

### REFERENCE TO AN ELECTRONIC SEQUENCE LISTING

The contents of the electronic sequence listing (PCTCN2023074352-seql.html; Size: 8.61 kb; and Date of Creation: Feb. 22, 2023) is incorporated herein by reference in its entirety.

### TECHNICAL FIELD

The present invention relates to the field of pharmaceutical and chemical engineering, and mainly relates to tumor weak acid microenvironment sensitivity aptamer triptolide conjugates; specifically, it relates to novel acid sensitivity aptamer triptolide conjugates and applications.

### BACKGROUND ART

Triptolide, also known as tripterygium lactone, or tripterygium lactone alcohol, is an epoxy diterpenoid lactone compound extracted from the roots, leaves, flowers and fruits of Celastraceae plant Tripterygium wilfordii. It constitutes the main active components of Tripterygium wilfordii extract together with wilfordine, wilforine, wilforgine, wilforrtrine, wilforzine, and wilfordsine. It is insoluble in water and soluble in methanol, dimethyl sulfoxide, anhydrous ethanol, ethyl acetate and chloroform. Triptolide has broad-spectrum antitumor activity, which can inhibit the proliferation of various tumor cells and induce apoptosis and autophagy. The antitumor activity of triptolide is superior to that of traditional anticancer drugs such as adriamycin and paclitaxel, and it can effectively inhibit the proliferation of tumor cells at very low concentration (2-10 ng/mL). In addition, triptolide can also be against tumor drug resistance, improve the sensitivity of tumor cells to other anti-tumor drugs, and combine with chemotherapy drugs and ionizing radiation to play a synergistic effect.

Cancer is now one of the major threats to people's lives. One of the differences between tumor cells and normal cells is that certain proteins are abnormally expressed in tumor cells. For example, mucin 1 (MUC1) is abnormally expressed on the membrane surface of breast cancer cells (MCF-7) and its drug-resistant cell lines, and nucleolin and epidermal growth factor receptor (EGFR) are also proteins that are abnormally expressed in tumor cells, with the former being abnormally expressed in the cell lines of triple-negative breast cancer MDA-MB-231, lung cancer A549, and colon cancer HCT116, and the latter being commonly used as a lung cancer therapeutic target.

Nucleic acid aptamers (aptamer) are single-stranded oligonucleotides that bind to a specific target using their own folded high-level structure, mainly a piece of DNA (deoxyribonucleic acid), RNA (ribonucleic acid), or modified DNA and RNA. Nucleic acid aptamers can be bound to the target molecule through the 3D conformational complementation, with similar affinity and specificity as the antibody, and have the advantages of good water solubility, low immunogenicity, easy to produce, low cost and good stability, and have been proven safe in clinical trials. Due to the specific delivery at the cellular level, nucleic acid aptamers have been widely used as targeting molecules in drug targeting delivery research. For example, Mucin 1 (MUC1) nucleic acid aptamer Apt can specifically bind to MUC1 with high affinity and its sequence is short, which is easy to be synthesized and purified. The aptamer AS1411 specifically binds to nucleolin, which is highly expressed on the surface of tumor cells, and at the same time, nucleolin on the membrane surface promotes macropinocytosis and increased uptake of AS1411 by tumor cells, whereas AS1411 enhances its stability by forming a G-quadruplex structure. The aptamer E07 can specifically bind to EGFR, the epidermal growth factor receptor.

Apt, AS1411, and E07 aptamers all have the characteristics of good water solubility, high stability and targeting highly expressed proteins in tumor cells, which can be used as carriers for drug transport. As a broad-spectrum anticancer drug, triptolide has strong antitumor effects, but it also has large toxic side effects and poor water solubility. Therefore, the modified triptolide is conjugated with an aptamer to transport triptolide to a specific tumor location by using the targeting function of the aptamer, which binds to the highly expressed receptor on the cell membrane, so as to make triptolide kill the tumor while avoiding the killing effect on normal cells.

In addition to the specific high expression of certain proteins by tumor cells, the lysosomal content of tumor tissues is much higher than that of normal tissues because tumor cells need to infiltrate surrounding tissues to achieve cell proliferation and metastasis. Lysosomes are important cellular degraders in eukaryotic cells to maintain the metabolic stability of the cell, and one of their characteristics is an acidic environment (pH=3.5-5.5) within the organelle.

In the prior art, the enol ether bond is used as an acidic linking bond to connect triptolide and the aptamer, and it was found during the study that the enol ether bond can be cleaved only at pH=4 or below. This resulted in a less efficient release of the aptamer triptolide conjugate formed through this linking bond in tumor cells and poor cancer cell inhibition.

### SUMMARY OF THE INVENTION

An object of the present invention is to overcome the defects that the linking bond in the aptamer triptolide conjugate existing in the prior art has low acid sensitivity and is not easy to cleave under the environment of cancer cells in vivo, resulting in insufficient directional release of triptolide, which in turn results in a lack of significant inhibition of the cancer cells, and to design a new linking bond to serve as a conjugating arm between triptolide and an aptamer, and, specifically, to provide a new type of acid-sensitive aptamer triptolide conjugate and application.

In order to realize the above mentioned objects of the invention, the present invention provides the following embodiments:

An acid-sensitive aptamer triptolide conjugate, having the following general formula:

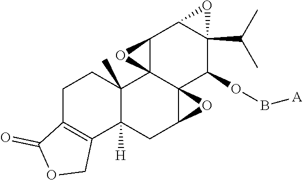

wherein, A is a nucleic acid aptamer; B is a linking bond linking triptolide to the nucleic acid aptamer, and said linking bond has an acetal ester functional group formed at the position 14 hydroxyl of triptolide.

The embodiment of the present invention discloses an aptamer triptolide conjugate, wherein the 14-position hydroxyl group of triptolide and the aptamer are conjugated by a linking bond (acetal ester functional group) formed by acetal and ester groups, which is an acid-sensitive linking bond with a cleaving condition of (pH=3.5-6.5), which is pH less sensitive and easier to cleave in the tumor microenvironment compared to the now existing enol ether linking bond (with a cleaving pH of less than pH 3.5). Based on the characteristics of the aptamer targeting the highly expressed proteins on the membrane surface of tumor cells, triptolide is delivered by the conjugate targeting to tumor cells and endocytosed into lysosomes. Based on the characteristics of the lysosomal acidic environment, the acetal linking bond releases the intact triptolide in the lysosomal acidic environment, and targets the tumor cells for killing.

An acid-sensitive aptamer triptolide conjugate, having the following general formula:

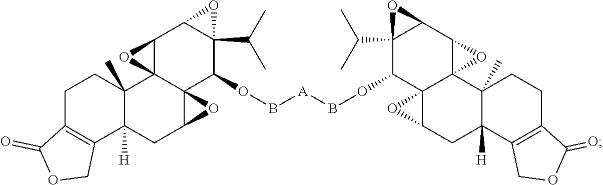

wherein A is a nucleic acid aptamer;
B is a linking bond linking triptolide to the nucleic acid aptamer, and said linking bond has an acetal ester functional group formed at the position 14 hydroxyl of triptolide. The conjugate is the conjugate of two triptolide to one nucleic acid aptamer.

As a preferred embodiment of the present invention, the linking bond B has a structure of the following general formula:

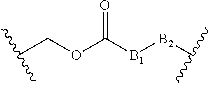

A study of the structure-activity relationship of triptolide found that its position 14 hydroxyl is the active site and also the modification site, and the linking bond B connects with the position 14 hydroxyl of triptolide to form an acetal linking bond, which can close the toxic effect of triptolide on normal tissues, and at the same time, it releases the intact triptolide in the lysosomal acidic environment, so as to achieve the effect of precise treatment of cancer.

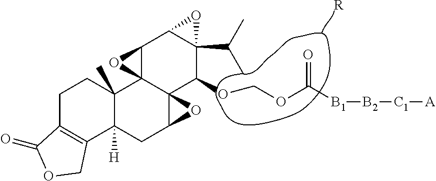

As shown in the above formula, the circled position is a new chemical bond formed by linking bond B with the hydroxyl group at position 14 of triptolide, which is defined herein as the R-bond that is acetal ester bond.

The following formula shows the reaction of this acid-sensitive aptamer triptolide conjugate.

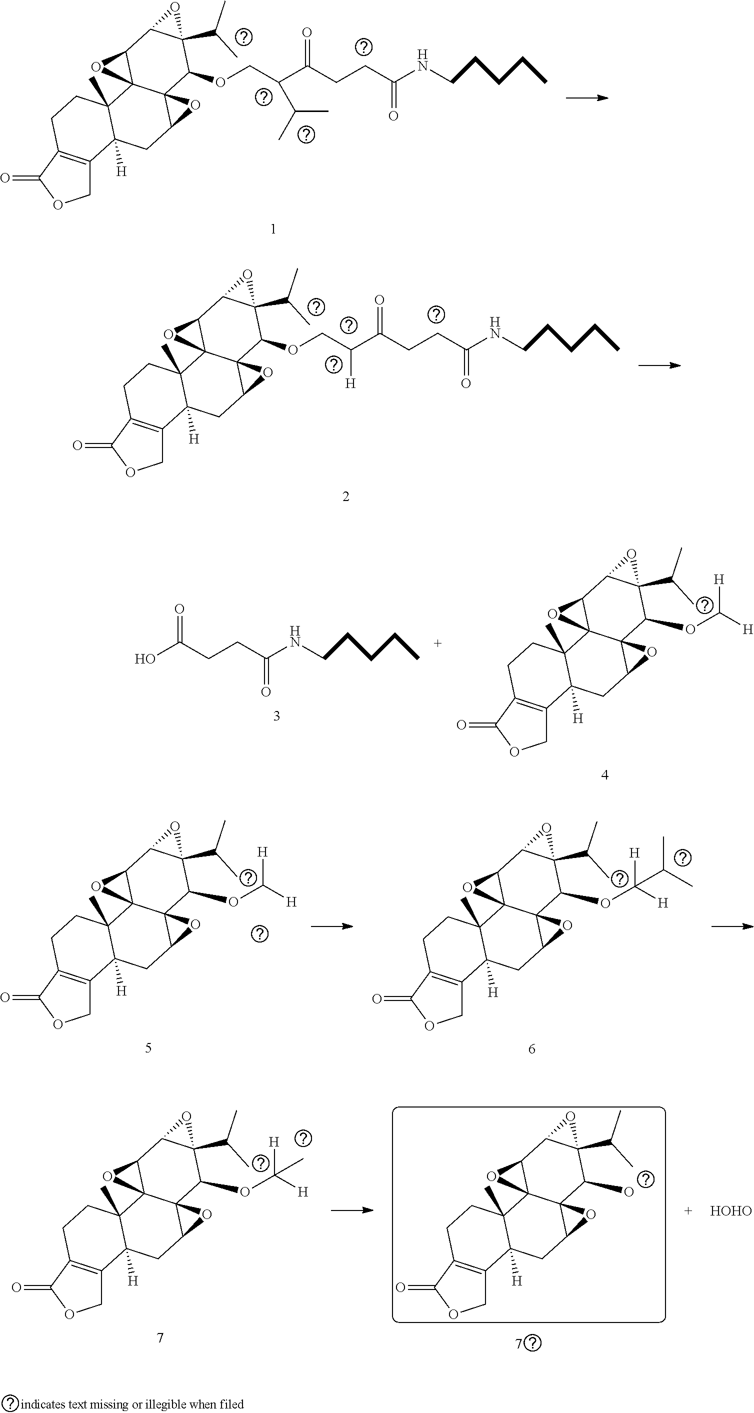

Cleavage mechanism: In an acidic environment, hydrogen ions attack the oxygen atom at the ester group end of the acetal ester bond of the aptamer triptolide conjugate (1), forming an unstable oxonium salt intermediate (2), leading to further cleavage to form the a portion containing the aptamer linking bond portion (3) and a highly unstable triptolide intermediate containing carbonium (4), and then a molecule of water binds to the carbonium of the triptolide intermediate (5), and the carbonium proton transfers to the oxygen atom in the water (6), and then the proton transfers to the oxygen atom of the triptolide, forming the unstable triptolide hemiacetal structure (7), and the carbon and oxygen bond in the hemiacetal structure cleaves to form triptolide and formaldehyde.

Further preferably, the structure of B₁ has the following general structure:

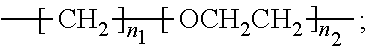

wherein 0≤n₁≤100, preferably, 0≤n₁≤50, more preferably, 0≤n₁≤20;
wherein 0≤n₂≤100, preferably, 0≤n₂≤50, more preferably, 0≤n₂≤20;
n₁ and n₂ take values in the range including 0, with the provision that they are not both 0;
specifically, n₁, n₂ preferably take values of 2, 3, 5, 10.

In this scheme, for B₁, wherein 0≤n₁(n₂)≤100, but the design and modification of the alkane chain or alkoxy chain can result in interfering with the binding of triptolide-aptamers to their target proteins, appropriately increasing the contact area of the linking bond with the tumor microenvironment, and increasing the rate of release of triptolide. However, the longer alkoxy chain is synthesized at a high cost and at a low yield, so it is more preferable that 123 n₁(n₂)≤20. n₁ and n₂ take values in the range including 0, with the provision that they are not both 0. Specifically, n₁, n₂ preferably take values of 2, 3, 5, 10.

As a preferred embodiment of the present invention, said nucleic acid aptamer is a modified nucleic acid aptamer, and said modified nucleic acid aptamer has the general structure of A₁-C₁ or C₁-A₁-C₁ wherein the structure of C₁ is:

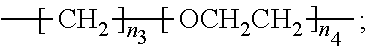

wherein 0≤n₃≤100, preferably, 0≤n₃≤50, more preferably, 0≤n₃≤20;
wherein 0≤n₄≤100, preferably, 0≤n₄≤50, more preferably, 0≤n₄≤20;
n₃ and n₄ take values in the range including 0, with the provision that they are not both 0;
specifically, n₃, n₄ preferably take values of 2, 3, 5, 10.

In this scheme, for C₁, wherein 0≤n₃(n₄)≤100, but the design and modification of the alkane chain or alkoxy chain can result in interfering with the binding of triptolide-aptamers to their target proteins, appropriately increasing the contact area of the linking bond with the tumor microenvironment, and increasing the rate of release of triptolide. However, the longer alkoxy chain is synthesized at a high cost and at a low yield, so it is preferred that 0≤n₃(n₄)≤50, and further preferred 0≤n₃(n4)≤20. n₃ and n₄ take values in the range including 0, with the provision that they are not both 0. Specifically, n₁, n₂ preferably take values of 2, 3, 5, 10.

As a preferred embodiment of the present invention, said conjugate is obtained by reacting a triptolide derivative with a nucleic acid aptamer modifier. Specifically, said triptolide derivative is obtained by reacting the hydroxyl group at the 14 position of triptolide in an organic solvent with the linking bond B. Said formation of triptolide derivatives with the hydroxyl group at the 14 position of triptolide contains a condensate ester functional group.

As a preferred embodiment of the present invention, said conjugate has a structure of the following general formula:

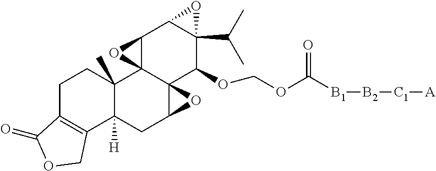

Said conjugates are obtained via the B₂ functional group on the triptolide derivative by any one of a substitution reaction, a cyclization reaction or an addition reaction, specifically, a substitution reaction between an amino group and a carboxyl group; or an addition reaction between a sulfhydryl group and a maleamide group, or a cyclization reaction between an azide group and an alkynyl group.

As a preferred embodiment for the present invention,
B₂ has the following general formula:

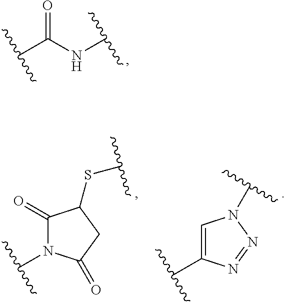

Said nucleic acid aptamer comprises any one of AS1411, Pegaptanib, Sgc8c, A10, DNA aptamer, RNA aptamer, CL4, Apt and E07.

The sequence of the nucleic acid aptamer is shown below:

AS1411:
(SEQ ID NO: 1)
GGTGGTGGTGGTTGTGGTGGTGGTGG;
Pegaptanib:
(SEQ ID NO: 2)
GCGAACCGAUGGAAUUUUUGGACGCUCGC;
Sgc8c:
(SEQ ID NO: 3)
ATCTAACTGCTGCGCCGCCGGGAAAATACTGTACGGTTAGA;
A10:
(SEQ ID NO: 4)
GGGAGGACGAUGCGGAUCAGCCAUGUUUACGUCACUCCUUGUCAAUCCUC
AUCGGCAGACGACUCGCCCGA;
DNA aptamer:
(SEQ ID NO: 5)
GGGAGACAAGAATAAACGCTCAA-(25N)-
TTCGACAGGAGGCTCACAACAGGC;
RNA aptamer:
(SEQ ID NO: 6)
GGGGGCAUACUUGUGAGACUUUUAUGUCACCCCC;
CL4:
(SEQ ID NO: 7)
GCCUUAGUAACGUGCUUUGAUGUCGAUUCGACAGGAGGC;
Apt:
(SEQ ID NO: 8)
GCAGTTGATCCTTTGGATACCCTGG;
E07:
(SEQ ID NO: 9)
GGACGGAUUUAAUCGCCGUAGAAAAGCAUGUCAAAGCCGGAACCGUCC.

As a preferred embodiment of the present invention, it is provided a method of preparing the acid-sensitive aptamer triptolide conjugate according to the present patent, specifically comprising the following steps to obtain the same:
Step 1: Preparation of a triptolide derivative, wherein the position 14 hydroxyl of triptolide is connected to an acetal ester functional group;
Step 2: Modification of a nucleic acid aptamer to obtain a modified nucleic acid aptamer modifier A₁-C₁;
Step 3: Linking the triptolide derivative with the nucleic acid aptamer modifier in Step 2 via a B₂ linker, to obtain the acid-sensitive aptamer triptolide conjugate.

Preferably, said acid-sensitive triptolide aptamer conjugate can be prepared by a person skilled in the art in conjunction with what is described in the present invention and attempts in the art.

The position 14 hydroxyl of triptolide is terminated by forming an acetal ester functional group with the linking bond to obtain the triptolide derivative; meanwhile, the aptamer is modified with an alkane or alkoxyalkane chain; then, according to the type of functional group of the end group of the triptolide derivative, the functional group of the end of the aptamer linking the alkane or alkoxyalkane chain is modified; finally, the triptolide derivative is bound to the modified triptolide derivative to obtain the triptolide derivative by using the amino group or sulfhydryl group or azide group on the nucleic acid aptamer and the carboxyl group or maleamide group or azide under the action of catalytic reagents. The two are bound to form the B₂ linkage described above.

Use of the acid-sensitive triptolide aptamer conjugate as described above in the preparation of an antitumor drug.

Beneficial effects of the present invention compared to the prior art are as follows.
1. In the aptamer triptolide conjugate provided by the present invention, the linking bond between triptolide and the aptamer has an acetal ester functional group. That is, the linking bond between the position 14 hydroxyl of the linking triptolide and the aptamer forms a linking bond with an acetal ester functional group. The linking bond is an acid-sensitive linking bond, which cleaves under the conditions of (pH=3.5-6.5), with lower pH sensitivity, better acid responsiveness, and easier cleavage in the tumor microenvironment.
2. In the aptamer triptolide conjugate provided by the present invention, in vitro activity study with AS1411-TP showed that AS1411-TP had comparable anti-tumor effect to triptolide on each tumor cell, and the connection of aptamer did not affect the anti-tumor effect of triptolide, and also proved that the tumor microenvironment-responsive acid-sensitive linking bond could be cleaved in the tumor cell, releasing the triptolide.
3. Regarding the aptamer triptolide conjugate provided by the present invention in a normal cellular environment, the aptamer closes the active group of triptolide, resulting in triptolide not being able to be released in its entirety, and the cytotoxicity of the conjugate is very low in comparison to simple triptolide, which indicates that the aptamer does not damage normal cells in the human body after it is entered into the human body. It is less toxic to the human body.
4. The aptamer triptolide conjugate provided by the present invention has better tumor targeting in vivo, while circumventing other tissues to achieve the effect of tumor targeting; intravenous administration experiments show that AS1411-TP has a better anti-tumor effect in vivo, and the linking bond used to link triptolide and the aptamer plays an obvious role in vivo, such that the conjugate does not cleave in normal tissues and has less toxicity; while it cleaves in tumor tissues and releases triptolide to apply therapeutic effects.

### BRIEF DESCRIPTION OF DRAWINGS

FIG. 1 shows the MS spectrum of compound 5;

FIG. 2 shows the MS spectrum of compound 7;

FIG. 3 shows the MS spectrum of compound 9;

FIG. 4 shows the HPLC analysis of the stability of the conjugate (AS1411-TP) in serum at different time points;

FIG. 5 shows the HPLC analysis of the stability of the conjugate (E07-TP) in serum at different time points;

FIG. 6 shows the HPLC analysis of the release of triptolide from the conjugate (AS1411-TP) at different pH values;

FIG. 7 shows the HPLC analysis of the release of triptolide from the conjugate (E07-TP) at different pH values;

FIG. 8 shows the HPLC analysis of the release of triptolide from the conjugate (AS1411-TP conjugate linked by an enol-ether bond) at different pH values;

FIG. 9 shows the chemical formula for AS1411-TP-1;

FIG. 10 shows a graph of the inhibitory effect of AS1411-TP, TP, and AS1411 on colon cancer cell line HCT116;

FIG. 11 shows a graph of the inhibitory effect of AS1411-TP, TP, and AS1411 on the lung adenocarcinoma cell line A549;

FIG. 12 shows a graph of the inhibitory effect of AS1411-TP, TP, and AS1411 on the breast cancer cell line MDA-MB-231;

FIG. 13 shows a graph of the inhibitory effect of AS1411-TP, TP, and AS1411 on the pancreatic cancer cell line PANC-1;

FIG. 14 shows a graph of the inhibitory effect of AS1411-TP, TP, and AS1411 on the hepatocellular carcinoma cell line HepG2;

FIG. 15 shows a graph of the toxic effect of AS1411-TP, TP, and AS1411 on human embryonic kidney cells HEK293;

FIG. 16 shows a graph of the toxic effect of AS1411-TP, TP, and AS1411 on human normal hepatocytes LO2;

FIG. 17 shows the distribution of AS1411-TP, TP, and AS1411 in different tissues of the tumor-bearing mice colon cancer model;

FIG. 18 shows the distribution of AS1411-TP, TP, and AS1411 in different tissues of the tumor-bearing mice breast cancer model;

FIG. 19 shows the distribution of AS1411-TP, TP, and AS1411 in different tissues of the tumor-bearing mice lung cancer model;

FIG. 20 shows a graph of AS1411-TP, TP, and AS1411 against mouse colon cancer xenograft tumors;

FIG. 21 shows a graph of AS1411-TP, TP, and AS1411 against mouse breast cancer xenograft tumors;

FIG. 22 shows a graph of AS1411-TP, TP, and AS1411 against mouse lung cancer xenograft tumors;

FIG. 23 shows a graph of the data analysis of the in vivo stability tests of AS1411-TP, TP, and AS1411.

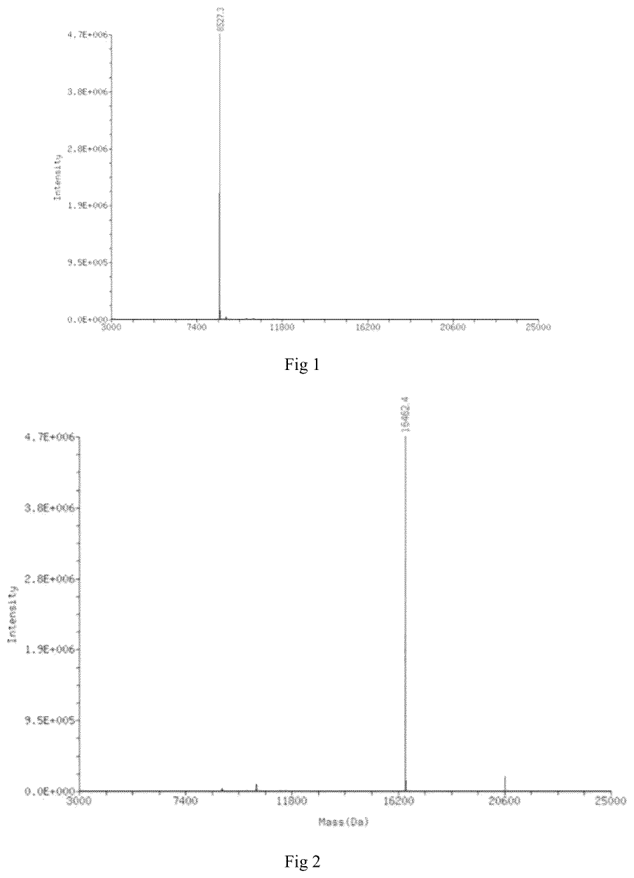

### DETAILED DESCRIPTION OF THE INVENTION

The present invention is described in further detail below in connection with test examples and specific embodiments. However, it should not be construed that the scope of the above subject matter of the present invention is limited to the following embodiments, and any technology realized based on the contents of the present invention is within the scope of the present invention.

EXAMPLE 1

This example provides an acid-sensitive aptamer triptolide conjugate, labeled Apt-TP, and the structural formula and reaction route of said conjugate are shown below:

Aptamer 1 is an amino-modified Apt synthesized by the Nucleic Acid Synthesizer AKTAOligopilot100system; specifically, Aptamer 1:
NH₂—(CH₂)₆-5′GCAGTTGATCCTTTGGATACCCTGG3′MS: 7843.

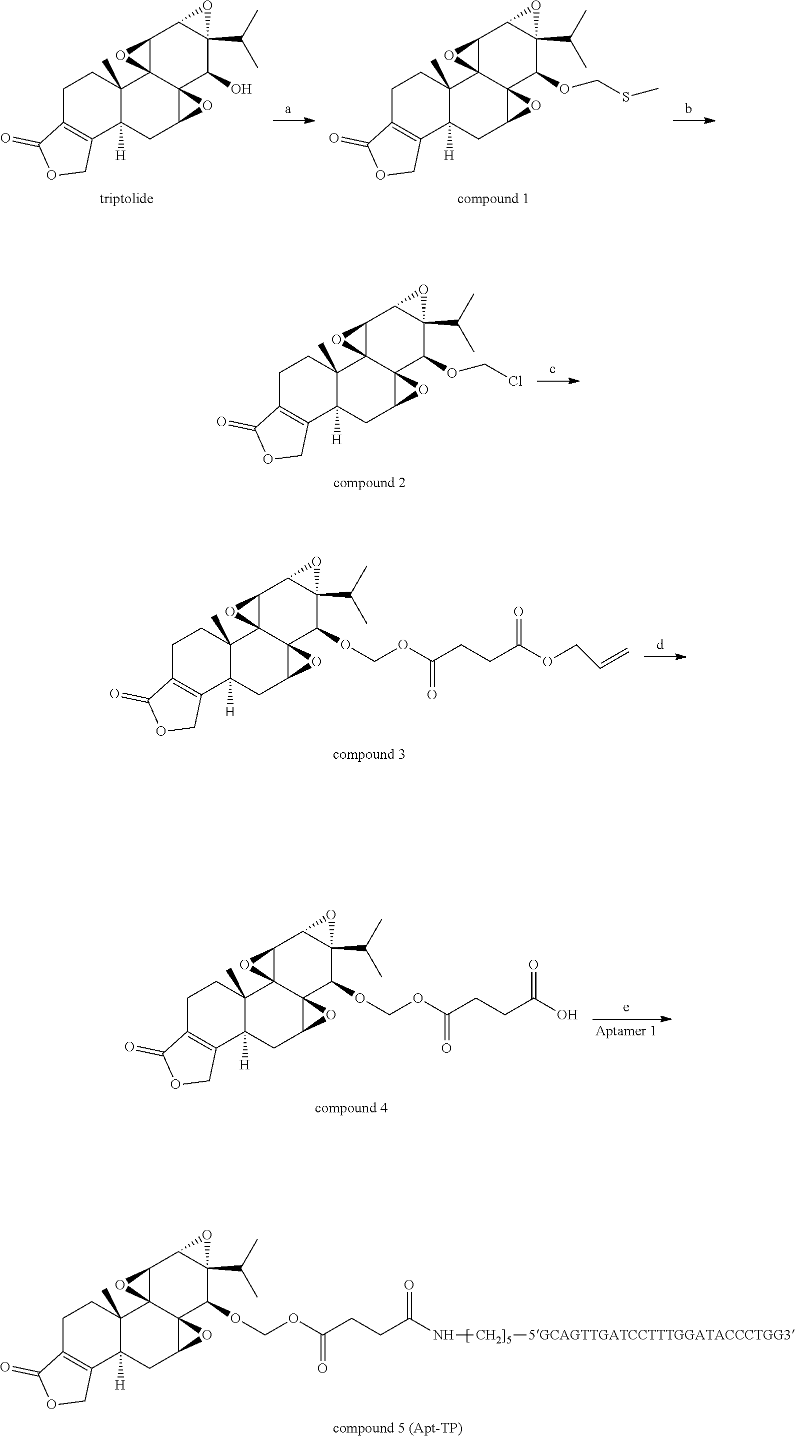

Specifically, a process for the preparation of compound 5 provided in Example 1 above is provided:

### Compound 1:

Under an ice bath, 1 eq of triptolide and 8 eq of dimethyl sulfide were dissolved in acetonitrile solution, 4 eq of benzoyl peroxide was added in four portions over 40 min, and the mixture was stirred under nitrogen protection for 1 h under an ice bath and then for 2 hours at room temperature. The reaction was complete as confirmed by thin-layer chromatography and LC-MS detection, and the mixture was diluted with ethyl acetate and washed with 10% Na₂CO₃ and saline. The organic phase was dried with anhydrous Na₂SO₄, filtered and concentrated to obtain the crude product, which was purified by silica gel column chromatography to obtain the pure white powder Compound 1 (MS: 420.2).

### Compound 2:

After dissolving 1 eq of Compound 1 in DCM solution, 1.5 eq SO₂Cl₂ was added and stirred at room temperature for 2 h and then the solvent was directly evaporated to give the crude Compound 2 (MS: 408.3) which was directly used in the next step.

### Compound 3:

1.0 eq of Compound 2 was dissolved in 20 ml of DCM solution, and 0.7 eq of 18-crown ether-6 and 1.7 eq of 4-(allyloxy)-4-oxobutanoic acid sodium were added and stirred at room temperature for 2 h under nitrogen protection. The reaction was complete as confirmed by thin-layer chromatography and LC-MS detection. Then DCM was added to dilute. Saturated sodium bicarbonate was added, and after collecting the organic phase, it was washed twice with saturated saline. The organic phase was dried with anhydrous Na₂SO₄, filtered and concentrated to obtain the crude product, which was purified by silica gel column chromatography to obtain Compound 3 (MS: 530.3).

### Compound 4:

1.0 eq of Compound 3 and 2.5 eq of 1,3-dimethylbarbituric acid were dissolved in a solution of dichloromethane and ethyl acetate in a volume ratio of 1:1, and then 0.1 eq of tetrakis(triphenylphosphine)palladium was added. The whole reaction system was kept at room temperature condition for 2 h. After the reaction, the organic solvent was removed under reduced pressure at 40° C., and the obtained crude product was purified by column chromatography to obtain Compound 4 (MS: 490.4).

### Compound 5:

The synthesized amino modified Apt (Aptamer 1) was taken 1 eq dissolved in deionized water, 100 eq of NaHCO₃ buffer with pH 9 was added, 100 eq of DMT-MM aqueous solution was added, and finally 70 eq of Compound 4 dissolved in DMSO was added. The reaction was shaken at 37° C. for 24 h, and after the reaction was complete as monitored by HPLC, 2.5 volumes of anhydrous ethanol, which was stirred and placed on dry ice for 1 h and then removed and centrifuged at high speed at low temperature. The precipitate was collected and dissolved into solution with dd-H₂O. Finally, the product was purified and collected by RP-HPLC and then lyophilized in a lyophilizer to obtain the pure Compound 5 (MS: 8527.3) as a white powder.

### EXAMPLE 2

This example provides an acid-sensitive aptamer triptolide conjugate, labeled E07-TP, and the structural formula and reaction route of said conjugate are shown below:

Aptamer 2 is sulfhydryl PEG-modified E07 synthesized by the nucleic acid synthesizer AKTAOligopilot100 system.

Specifically, the Aptamer 2:
SH—CH₂CH₂—(OCH₂CH₂)₁₀—O-5′GGACGGAUUUAAUCGCCGUAGAAAAGCAUGUCAAAGCCGGAACCGUCC3′MS: 15990.5.

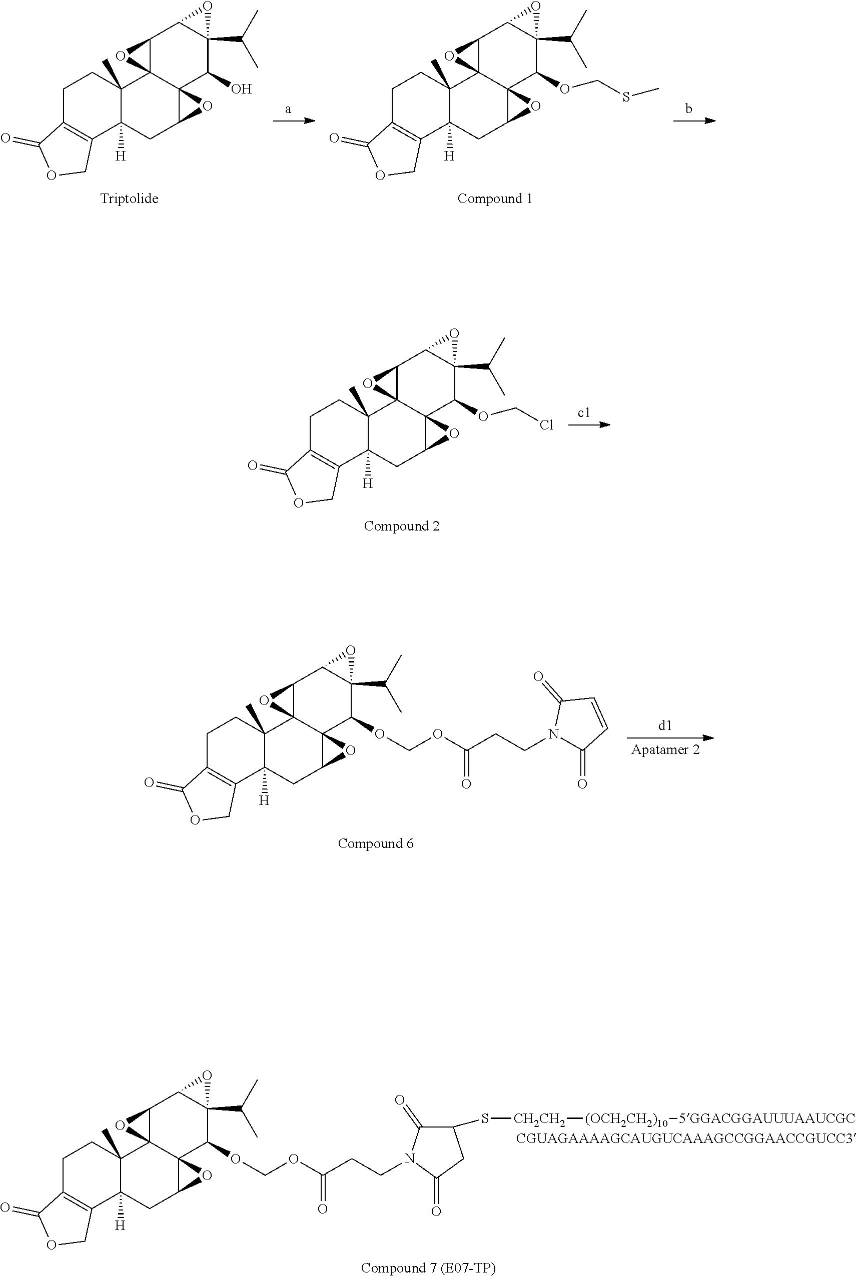

Among others, the process of preparing the conjugate 7 comprises the following steps: Compound 1 and Compound 2 were prepared in the same manner as in Example 1 and are not described herein.

### Compound 6:

1.0 eq of Compound 2 was dissolved in DCM solution, 0.7 eq of 18-crown ether-6 and 2 eq of sodium 3-maleimidopropionate were added, and the reaction was stirred at room temperature for 2 h under nitrogen protection, and the reaction was complete as confirmed by thin-layer chromatography and LC-MS detection. Then DCM was added to dilute. Saturated sodium bicarbonate was added, and after collecting the organic phase, it was washed twice with saturated saline, and the organic phase was dried with anhydrous Na₂SO₄, filtered and concentrated to obtain the crude product, which was purified by silica gel column chromatography to obtain the Compound 6 (MS: 541.2).

### Compound 7:

1 eq of sulfhydryl modified E07 (Aptamer 2) was dissolved in a buffer of sodium carbonate and sodium bicarbonate at pH=7.4, activated by the addition of 50 eq of TECP, and the reaction was carried out by shaking at 4° C. for 2 h, then it was added to the DMF solution wherein 100 eq of Compound 6 was dissolved, and the reaction system was kept at room temperature for 24 h. At the end of the reaction, 2.5-fold amount of ethanol was used to precipitate, and the crude product was purified by RP-HPLC to give the target product Compound 7 (MS: 16462.4).

### EXAMPLE 3

This example provides an acid-sensitive aptamer triptolide conjugate, labeled AS1411-TP, and the structural formula and reaction route of said conjugate are shown below:

Aptamer 3 is azide PEG-modified AS1411 synthesized by the nucleic acid synthesizer AKTAOligopilot100system; Aptamer 3 is the
N₃—CH₂CH₂—(OCH₂CH₂)₃—O-5GGTGGTGGTGGTTGTGGTGGTGGTGG3′MS: 8553.4

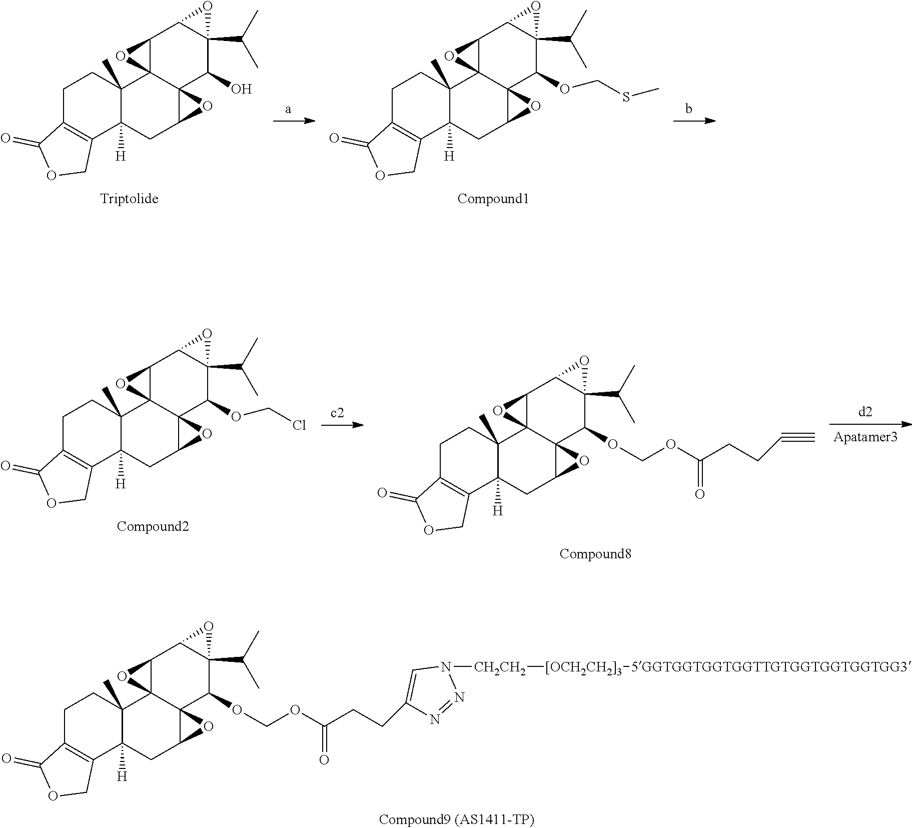

Among others, the process of preparing the conjugate 7 comprises the following steps:

Compound 1 and Compound 2 were prepared in the same manner as in Example 1 and are not described herein.

### Compound 8:

1.0 eq of Compound 2 was dissolved in DCM solution, 0.7 eq of 18-crown ether-6 and 3 eq of sodium 4-pentynoate were added and stirred at room temperature for 2 h under nitrogen protection, and the reaction was complete as confirmed by thin-layer chromatography and LC-MS detection. Then DCM was added to dilute. Saturated sodium bicarbonate was added, and after collecting the organic phase, it was washed twice with saturated saline. The organic phase was dried with anhydrous Na₂SO₄, filtered and concentrated to obtain the crude product, which was purified by silica gel column chromatography to obtain the Compound 8 (MS: 470.4).

### Compound 9:

1 eq of azide PEG modified Aptamer 3 was dissolved in a buffer of sodium carbonate and sodium bicarbonate at pH=7.4, 3 eq of triethylamine and an equal volume of tert-butanol was added to a DMF solution dissolved with 100 eq of Compound 8, then 50 eq of CuSO_{4.}5H₂O and 50 eq of sodium ascorbate were added, and the reaction system was reacted for 24 hours at room temperature under nitrogen protection. After the reaction ends, 2.5 times amount of ethanol was used to precipitate and the crude product was purified by RP-HPLC to give the target product Compound 9 (MS: 9023.4).

The MS spectra of the conjugates prepared in Examples 1-3 are shown in FIGS. 19-21.

### I. Serum Stability and Weak Acid Microenvironment Sensitivity Studies of Conjugates

In order to verify the serum stability and weak acid microenvironment sensitivity of the conjugates of the present invention, HPLC assays were performed, respectively, as described below:

Materials: AS1411-TP conjugate (acetal ester bond linkage), E07-TP conjugate (acetal ester bond linkage), AS1411-TP-1 conjugate (enol ether bond linkage), mouse serum was obtained from BALB/c mice, female, 7-8 weeks age (SPF(Beijing) Biotechnology Co. Ltd.)

Experimental protocol: 2 mg of AS1411-TP conjugate (acetal ester bond linkage) and E07-TP conjugate (acetal ester bond linkage) were dissolved in 100 μL of mouse serum, which was used to simulate the stabilization of the conjugates in the blood circulatory system, and 6 groups were set up in parallel. The drug and serum were incubated in an incubator at 37° C. The drug was filtered after 0 h, 1 h, 2 h, 4 h, 12 h and 24 h, respectively, and the content of the conjugate was determined by HPLC after different times of dissolution. The test results are shown in FIGS. 4 and 5. As can be seen from FIGS. 4 and 5, only the conjugate was present in the HPLC graphs within 24 h, and there was no decomposition of the conjugate, which indicates that the aptamer-triptolide conjugate (acetal bond linkage) in the present invention can be stable in serum for a long period of time.

The tumor microenvironment is a weakly acidic environment and the sensitivity of the conjugate to the weakly acidic microenvironment determines the therapeutic effect of the drug. 2 mg of AS1411-TP-1 conjugate (allyl ether bond linkage), AS1411-TP conjugate (acetal ester bond linkage), E07-TP conjugate (acetal ester bond linkage) were taken and dissolved in 100 μL of PBS buffer with different pH, which were used to simulate the tumor microenvironment, and the changes in the content of the conjugates in the different pH conditions were determined by HPLC respectively after 2 h. The results showed that the conjugates are sensitive to the weak acidic microenvironment, which is a weak acidic environment. Among them, AS1411-TP conjugate (acetal ester bond linkage) was used as a control group, named AS1411-TP-1, and the chemical formula of AS1411-TP-1 is shown in FIG. 9.

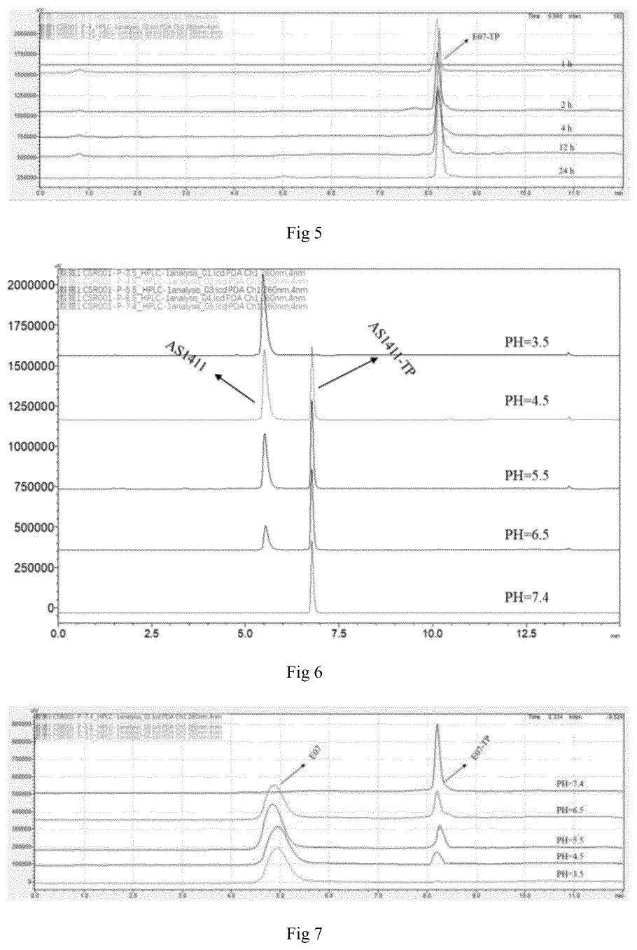

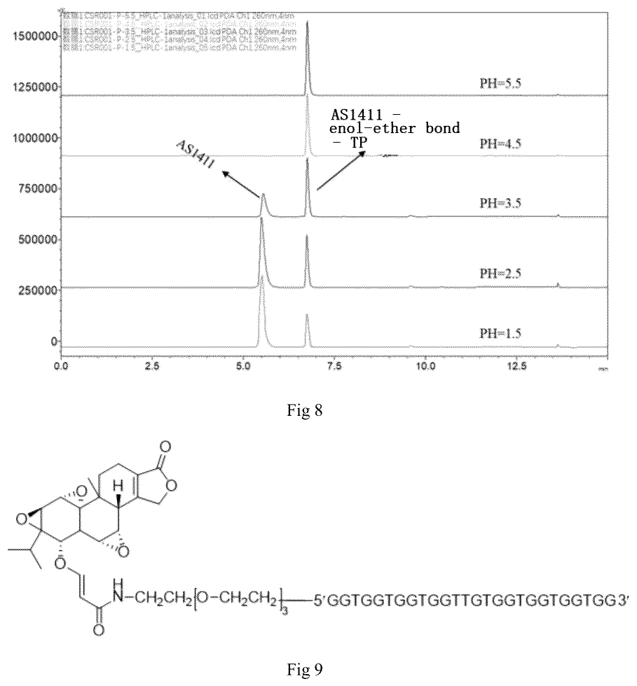

The results of the assay are shown in FIG. 6, FIG. 7 and FIG. 8, FIG. 6 shows the measurement result of HPLC of AS1411-TP conjugate, FIG. 7 shows the measurement result of HPLC of E07-TP conjugate, and FIG. 8 shows the measurement result of HPLC of AS1411-TP-1. It can be seen from the above three graphs that the conjugate connected through the acetal ester bond started to cleave at pH 6.5, and pH=3.5 when the conjugate was complete as cleaved, while the conjugate connected through the enol ether bond started to cleave only at pH 3.5, which proved that the acetal ester bond was more sensitive to the pH required for cleaving compared to the enol ether bond. It was easier to cleave in the tumor microenvironment to release the triptolide, which proved that the conjugate connected through the acetal bond had a better responsiveness to the tumor's weak acidic microenvironment.

The above serum stability and weak acid microenvironment sensitivity experiments fully demonstrate that the acetal ester bond in the conjugate in the present invention not only possesses serum stability, but at the same time, the bond cleaving requirement is lower in the acidic environment, and when the pH value is about 6, the linking bond cleaving can be achieved, and triptolide can be released, and the medicinal effect can be exerted. The release rate of triptolide was greatly improved, enabling more efficient utilization of triptolide.

Nucleolin aptamer AS1411 is a highly water-soluble, single-stranded oligonucleotide that can specifically bind to nucleolin proteins expressed at high levels on the membrane surface of tumor cells. Therefore, using the nucleolin aptamer-triptolide conjugate in Example 3 as an example, the in vitro activity, in vitro toxicity, in vivo distribution, in vivo activity, and in vivo stability of the conjugate of the present invention were investigated to fully characterize and validate the performance of the linking bond in the conjugate and the feasibility of inhibiting cancer cells in vivo.

### II. In Vitro Activity Study of AS1411-TP

Material: aptamer triptolide conjugate (AS1411-TP). Triptolide (TP) was provided by Shanghai Yaji Biotechnology Co. Ltd; aptamer (AS1411) was obtained from Chengdu Pilot Drug Development Co. Ltd; trypsin, thiazolyl blue (MTT) (Biofroxx, Germany); dimethyl sulfoxide (DMSO) (MP Biomedicals, France); fetal bovine serum (FBS) (Newzerum, New Zealand); DMEM medium, RPMI1640 medium, Petri dishes, 96-well plates (Corning, USA); colon cancer cells HCT116, breast cancer cells MDA-MB-231, non-small-cell lung cancer cells A549, pancreatic cancer cells PANC-1, and hepatocellular carcinoma cells HepG2 were purchased from the Shanghai Cell Bank of the Chinese Academy of Sciences and preserved in the laboratory.

### Experimental Protocol

### 2.1. Cell Resuscitation

Take the tumor cells HCT116, MDA-MB-231, A549, PANC-1 and HepG2 cell lines from the liquid nitrogen tank, place them in a 37° C. water bath, wait for the frozen cells to melt into cell suspension, centrifuge the cells at 1000 r/min for 3 min, discard the supernatant, add 1 ml of fresh complete medium containing 10% fetal bovine serum and 1% double antibody to mix the cells, then add 5 ml of medium, gently and repeatedly blowing the cells with a pipette gun to make them mix well. Then add 5 ml of medium, blow the cells gently and repeatedly with a pipette gun to mix them well, and finally transfer the cell suspension to a 10 mm dish and label the cell name, cell number, date of resuscitation and the operator's name, and place it in a cell culture incubator for cultivation. (HCT116, PANC-1 and HepG2 cell lines were cultured in DMEM medium, while MDA-MB-231 cell line and A549 cell line were cultured in RPMI1640 medium).

### 2.2 Cell Passaging

Take HCT116, MDA-MB-231, A549, PANC-1, HepG2 cells with good growth status, remove the medium, add 1 ml of sterile PBS to wash, add 1.5 ml of trypsin to digest the cells, about 2 min later the cells become round and fall off, add 1.5 ml of medium to terminate the digestion, transfer it to centrifugal tube, centrifuge at 1000 r/min for 3 min, discard the supernatant, add 1 ml of fresh medium to resuspend, then add fresh medium for 1:3 distribution to other culture dishes, and label the date of transmission and the number of generations. Place in the cell culture incubator for cultivation.

### 2.3 MTT Assay to Determine AS1411-TP Activity

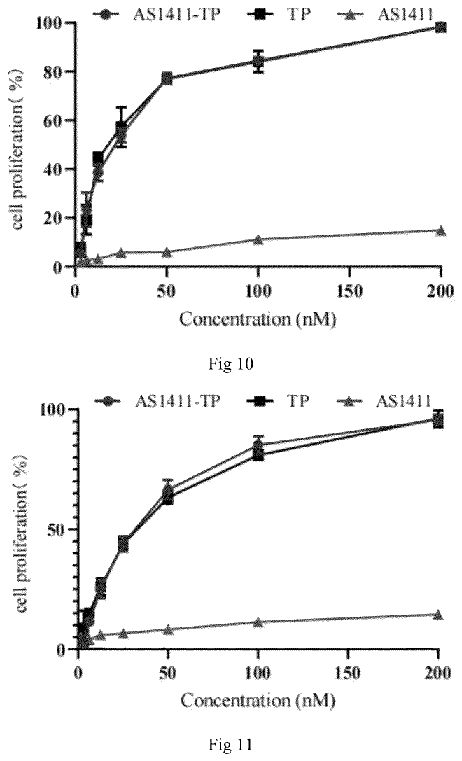

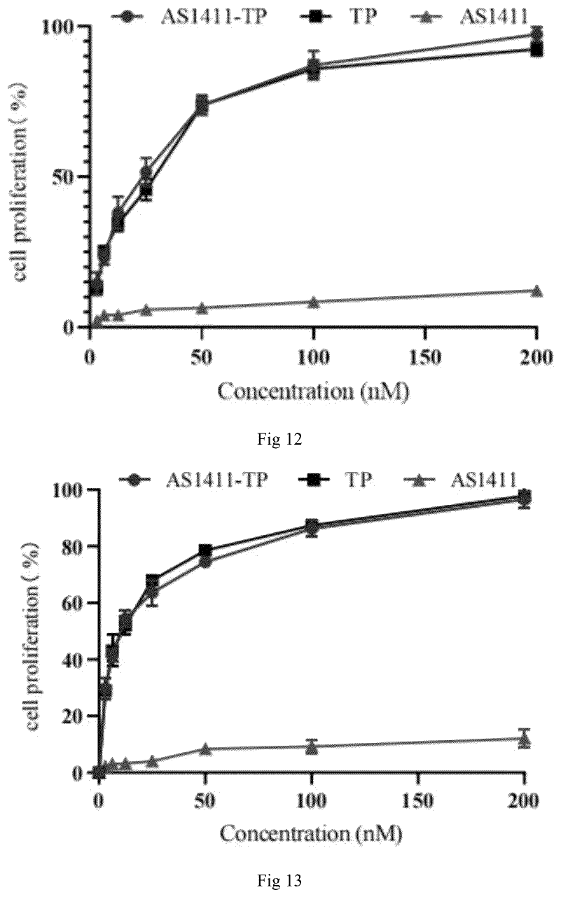

Take HCT116, MDA-MB-231, A549, PANC-1, HepG2 cells with good growth status, by the same operation as the cell passaging experiment, after cell resuspension, cell counting with hemocyte counting plate, with 2500 cells/well, plant in 96-well plate, after 24 h of cell culture, remove the original medium, add medium containing different concentrations of drugs and continue to cultivate for 48 hours. After 24 h of cell culture, the original medium was removed and the medium containing different concentrations of drugs was added to continue the culture for 48 hours. After the drug action, remove the original medium, add 100 μl of fresh medium, add 10 μl of MTT solution, incubate in the incubator for 2 h, add 150 ml of DMSO, incubate on shaking bed for 15 min, at 490 nm, measure the OD value by enzyme labeling instrument, and calculate the cell proliferation inhibition rate. The above steps were repeated three times. The inhibition profiles of AS1411-TP on the above five cancer cell lines are shown in FIGS. 10-14, and the experimental data are shown in Tables 1-5.

Table 1 summarizes the data of cell proliferation inhibition rate of AS1411-TP, TP, and AS1411 after 48 h of action on colon cancer cell line HCT116:

| concentration | AS1411-TP | | | TP | | | AS1411 | | |
|---|---|---|---|---|---|---|---|---|---|
| (nM) | sample 1 | sample 2 | sample 3 | sample 1 | sample 2 | sample 3 | sample 1 | sample 2 | sample 3 |
| 200.0 | 98.0396 | 99.3943 | 97.6337 | 97.2050 | 98.8058 | 98.5677 | 16.9608 | 13.5881 | 14.2937 |
| 100.0 | 83.9445 | 83.8214 | 79.5710 | 82.9707 | 80.3075 | 88.8251 | 10.6504 | 12.1637 | 10.9076 |
| 50.0 | 78.0639 | 74.8384 | 78.9549 | 75.5572 | 79.2127 | 76.2156 | 6.5424 | 6.5952 | 5.0330 |
| 25.0 | 56.0137 | 50.5940 | 54.8405 | 54.9296 | 66.2973 | 50.7426 | 5.4987 | 6.2971 | 5.6986 |
| 12.5 | 35.5002 | 38.0847 | 42.1452 | 42.1339 | 43.4099 | 47.0957 | 4.1987 | 3.0229 | 2.5102 |
| 6.3 | 22.9707 | 16.8472 | 30.6601 | 24.6824 | 20.0440 | 13.1188 | 2.9669 | 1.6683 | 3.1584 |
| 3.1 | 4.2221 | 5.5731 | 7.0957 | 9.8517 | 6.7785 | 3.8779 | 0.5424 | 1.4343 | 3.3666 |

Table 2 summarizes the data on the cell proliferation inhibition rate of AS1411-TP, TP, and AS1411 on the lung adenocarcinoma cell line A549 after 48 h of action:

| concentration | AS1411-TP | | | TP | | | AS1411 | | |
|---|---|---|---|---|---|---|---|---|---|
| (nM) | sample 1 | sample 2 | sample 3 | sample 1 | sample 2 | sample 3 | sample 1 | sample 2 | sample 3 |
| 200.0 | 93.5080 | 97.3771 | 96.2770 | 99.2234 | 97.0057 | 96.3263 | 14.5704 | 11.4171 | 14.3285 |
| 100.0 | 88.9080 | 81.4857 | 84.9945 | 80.0609 | 84.2971 | 83.2186 | 11.4727 | 11.8492 | 10.6369 |
| 50.0 | 69.8111 | 67.5314 | 62.1828 | 63.0205 | 66.0800 | 68.6718 | 9.0069 | 8.0722 | 7.6624 |
| 25.0 | 42.0455 | 42.0229 | 47.6989 | 41.4828 | 43.2000 | 46.9811 | 6.8129 | 6.6467 | 6.1306 |
| 12.5 | 20.6297 | 26.9486 | 27.6582 | 22.1328 | 29.4400 | 25.9563 | 5.5843 | 7.7330 | 4.4586 |
| 6.3 | 11.9297 | 10.7771 | 11.5908 | 13.6677 | 16.7429 | 15.2571 | 5.6975 | 3.3829 | 2.3885 |
| 3.1 | 4.1519 | 0.5029 | 4.9612 | 5.6673 | 3.5657 | 16.9811 | 3.2333 | 4.9611 | 2.2994 |

Table 3 summarizes the data on the inhibition of cell proliferation after 48 h of action of AS1411-TP, TP, and AS1411 on the breast cancer cell line MDA-MB-231:

| concentration | AS1411-TP | | | TP | | | AS1411 | | |
|---|---|---|---|---|---|---|---|---|---|
| (nM) | sample 1 | sample 2 | sample 3 | sample 1 | sample 2 | sample 3 | sample 1 | sample 2 | sample 3 |
| 200.0 | 99.9080 | 97.4857 | 94.9945 | 98.0609 | 94.2971 | 93.2186 | 14.4727 | 11.8492 | 10.6369 |
| 100.0 | 89.8111 | 87.5314 | 82.1828 | 83.0205 | 86.0800 | 88.6718 | 9.0069 | 8.0722 | 8.6624 |
| 50.0 | 72.0455 | 72.0229 | 77.6989 | 71.4828 | 73.2000 | 76.9811 | 6.8129 | 6.6467 | 6.1306 |
| 25.0 | 50.6297 | 56.9486 | 47.6582 | 42.1328 | 49.4400 | 45.9563 | 5.5843 | 7.7330 | 4.4586 |
| 12.5 | 31.9297 | 40.7771 | 41.5908 | 31.6677 | 36.7429 | 35.2571 | 4.6975 | 5.3829 | 2.3885 |
| 6.3 | 24.1519 | 20.5029 | 24.9612 | 25.6673 | 23.5657 | 26.9811 | 3.2333 | 4.9611 | 4.2994 |
| 3.1 | 10.2234 | 17.0057 | 16.3263 | 14.5704 | 11.4171 | 14.3285 | 2.2910 | 2.1209 | 2.5159 |

Table 4 summarizes the data of cell proliferation inhibition rate of AS1411-TP, TP, and AS1411 on pancreatic cancer cell line PANC-1 after 48 h of action:

| concentration | AS1411-TP | | | TP | | | AS1411 | | |
|---|---|---|---|---|---|---|---|---|---|
| (nM) | sample 1 | sample 2 | sample 3 | sample 1 | sample 2 | sample 3 | sample 1 | sample 2 | sample 3 |
| 200.0 | 98.1506 | 99.4049 | 96.2777 | 93.0451 | 98.8589 | 97.2313 | 9.6236 | 10.2679 | 9.9457 |
| 100.0 | 86.2970 | 89.0743 | 82.6857 | 80.3154 | 81.0105 | 79.6202 | 8.3147 | 7.1285 | 8.7216 |
| 50.0 | 72.5975 | 80.4612 | 71.5293 | 64.8389 | 65.1441 | 74.5337 | 7.2954 | 7.9552 | 9.6253 |
| 25.0 | 63.6216 | 69.1150 | 66.3683 | 58.9929 | 58.7555 | 59.2302 | 3.0417 | 4.2455 | 4.1946 |
| 12.5 | 56.5717 | 54.4015 | 55.4866 | 49.1929 | 47.8196 | 50.5663 | 3.6656 | 3.5639 | 3.6148 |
| 6.3 | 40.8545 | 39.1590 | 40.0068 | 35.9546 | 37.1753 | 34.7338 | 4.7169 | 1.9871 | 3.3520 |
| 3.1 | 30.7291 | 24.3744 | 22.5517 | 31.1631 | 30.2815 | 32.0448 | 0.3764 | 2.8325 | 3.9457 |

Table 5 summarizes the data of cell proliferation inhibition rate of AS1411-TP, TP, and AS1411 after 48 h of action on hepatocellular carcinoma cell line HepG2:

| concentration | AS1411-TP | | | TP | | | AS1411 | | |
|---|---|---|---|---|---|---|---|---|---|
| (nM) | sample 1 | sample 2 | sample 3 | sample 1 | sample 2 | sample 3 | sample 1 | sample 2 | sample 3 |
| 200.0 | 99.7291 | 94.3744 | 95.5517 | 95.1631 | 99.2815 | 99.0448 | 10.3764 | 15.8325 | 9.9457 |
| 100.0 | 87.8942 | 87.8467 | 82.8705 | 88.1738 | 88.3181 | 85.8901 | 7.2906 | 11.7328 | 8.7216 |
| 50.0 | 73.6283 | 75.2730 | 74.4507 | 78.5402 | 79.9729 | 77.1075 | 8.4300 | 6.9712 | 9.6253 |
| 25.0 | 61.8786 | 68.7080 | 60.2933 | 67.9620 | 66.7752 | 69.1489 | 4.1258 | 3.9268 | 4.1946 |
| 12.5 | 51.5632 | 57.2601 | 54.4117 | 48.6758 | 51.8210 | 55.5307 | 3.5280 | 2.4483 | 3.6148 |
| 6.3 | 39.1251 | 43.2621 | 41.1936 | 39.6846 | 39.8542 | 49.5151 | 1.8820 | 3.7911 | 3.3520 |
| 3.1 | 33.6216 | 29.1150 | 26.3683 | 28.9929 | 28.7555 | 29.2302 | 2.0417 | 2.2455 | 2.1946 |

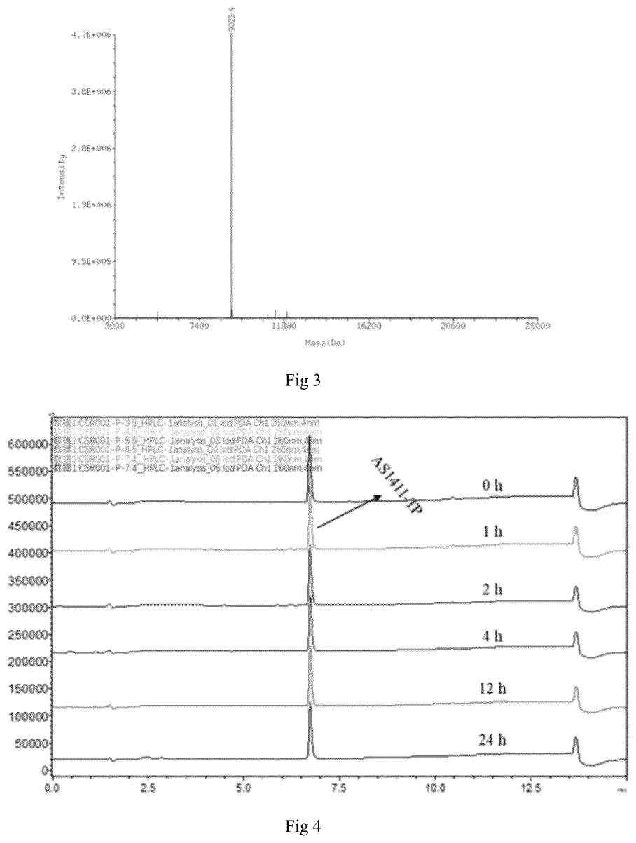

Combining the data in Tables 1-5 above and the statistical line graphs of the inhibitory effects of the drugs on different cancer cells shown in FIGS. 3-7, it can be seen that: the IC₅₀s of triptolide against colon cancer cells HCT116, breast cancer cells MDA-MB-231, lung adenocarcinoma cells A549, pancreatic cancer cells PANC-1, and hepatocellular carcinoma cells HepG2 were 18.88±0.13 nM, 22.38±0.24 nM, 30.23±0.17 nM, 14.4±0.09 nM, and 10.46±0.33 nM, respectively; and the IC₅₀s of the AS1411-TP pair against colon cancer cell HCT116, breast cancer cell MDA-MB-231, lung adenocarcinoma cell A549, pancreatic cancer cell PANC-1, and hepatocellular carcinoma cell HepG2 were 20.01±0.12 nM, 20.03±0.27 nM, 29.87±0.16 nM, 11.18±0.03 nM, and 11.21±0.26 nM, respectively. At the experimental concentration, AS1411 did not have any significant inhibitory effect on each tumor cell. The above data proved that AS1411-TP had comparable anti-tumor effect to triptolide on all tumor cells and the effect was not attributed to AS1411, indicating that the connection of aptamer did not affect the anti-tumor effect of triptolide, which also proved that tumor microenvironment-responsive acid-sensitive linking bonds could be cleaved in tumor cells to release triptolide.

### III. In Vitro Toxicity study of AS1411-TP

The same materials as those in Part II above are not repeated here, in addition to the following: human normal hepatocytes LO2, human embryonic kidney cells HEK293 were purchased from the Shanghai Cell Bank of the Chinese Academy of Sciences and preserved in our laboratory.

### Experimental Protocol

### 3.1 Cell Resuscitation

Take the tumor cells LO2 and HEK293 cell lines from the liquid nitrogen tank, place them in a 37° C. water bath, wait for the frozen cells to melt into cell suspension, centrifuge the cells at 1000 r/min for 3 min, discard the supernatant, add 1 ml of fresh complete medium containing 10% fetal bovine serum and 1% double antibody to mix the cells, then add 5 ml of medium, gently and repeatedly blowing the cells with a pipette gun to make them mix well. Then add 5 ml of medium, blow the cells gently and repeatedly with a pipette gun to mix them well, and finally transfer the cell suspension to a 10 mm dish and label the cell name, cell number, date of resuscitation and the operator's name, and place it in a cell culture incubator for cultivation. (HEK293 cell line was cultured with DMEM medium, LO2 cell line was cultured with RPMI1640 medium)

### 3.2 Cell Passaging

Take LO2 and HEK293 cells with good growth status, remove the medium, add 1 ml of sterile PBS to wash, add 1.5 ml of trypsin to digest the cells, about 2 min later the cells become round and fall off, add 1.5 ml of medium to terminate the digestion, transfer it to centrifugal tube, centrifuge at 1000 r/min for 3 min, discard the supernatant, add 1 ml of fresh medium to resuspend, then add fresh medium for 1:3 distribution to other culture dishes, and label the date of transmission and the number of generations. Place in the cell culture incubator for cultivation.

### 3.3 MTT Assay to Determine the Cytotoxicity of Aptamer-triptolide Conjugates

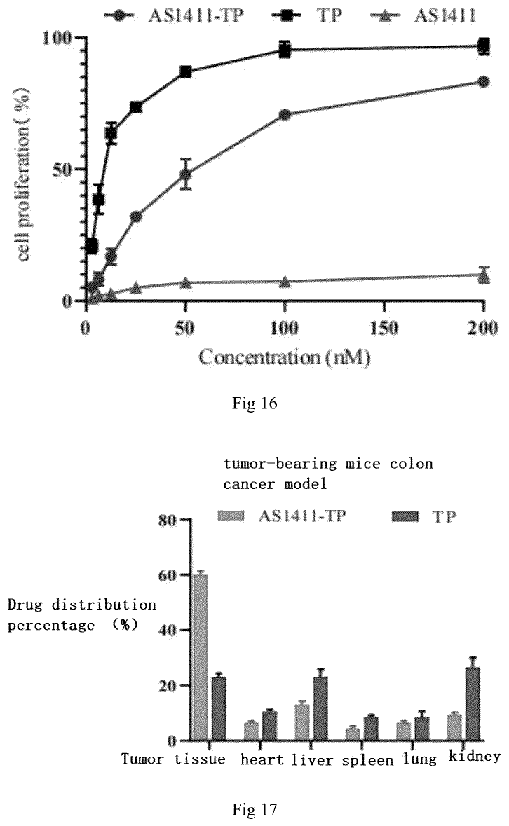

Take LO2 and HEK293 cells with good growth status, by the same operation as the cell passaging experiment, after cell resuspension, cell counting with hemocyte counting plate, with 3000 cells/well, plant in 96-well plate, after 24 h of cell culture, remove the original medium, add medium containing different concentrations of drugs and continue to cultivate for 48 hours. After 24 h of cell culture, the original medium was removed and the medium containing different concentrations of drugs was added to continue the culture for 48 hours. After the drug action, remove the original medium, add 100 μl of fresh medium, add 10 μl of MTT solution, incubate in the incubator for 2 h, add 150 ml of DMSO, incubate on shaking bed for 15 min, at 490 nm, measure the OD value by enzyme labeling instrument, and calculate the cell proliferation inhibition rate. The above steps were repeated three times. The toxicity profiles of AS1411-TP on normal hepatocytes LO2 and normal human embryonic kidney cells HEK293 are shown in FIGS. 15-16, and the experimental data are shown in Tables 6-7.

Table 6 shows the data of cell proliferation inhibition rate of AS1411-TP, TP, and AS1411 on human embryonic kidney cells HEK293 after 48 h action:

| concentration | AS1411-TP | | | TP | | | AS1411 | | |
|---|---|---|---|---|---|---|---|---|---|
| (nM) | sample 1 | sample 2 | sample 3 | sample 1 | sample 2 | sample 3 | sample 1 | sample 2 | sample 3 |
| 200.0 | 87.6596 | 89.6170 | 89.1489 | 98.2979 | 95.3901 | 100.6383 | 13.0496 | 11.5603 | 7.0200 |
| 100.0 | 79.2199 | 80.7801 | 81.4894 | 99.9291 | 94.3972 | 99.2908 | 9.5603 | 9.9858 | 6.6667 |
| 50.0 | 56.5937 | 43.2872 | 47.9317 | 89.5322 | 81.0372 | 89.5322 | 5.9341 | 4.5183 | 7.0200 |
| 25.0 | 29.6553 | 22.7331 | 23.1025 | 79.5322 | 73.0686 | 75.7495 | 4.6088 | 4.6251 | 5.5956 |
| 12.5 | 15.7481 | 15.4323 | 19.3525 | 69.2736 | 70.4830 | 71.1686 | 2.3262 | 2.9846 | 3.9285 |
| 6.3 | 8.5231 | 7.4099 | 9.3525 | 59.9053 | 53.1149 | 51.3265 | 1.4844 | 1.0633 | 2.0955 |
| 3.1 | 5.2744 | 4.1690 | 4.4492 | 35.0792 | 30.4489 | 32.7054 | 2.8090 | 1.3266 | 1.6489 |

Table 7 shows the data of cell proliferation inhibition rate of AS1411-TP, TP, and AS1411 on human normal hepatocytes LO2 after 48 h action:

| concentration | AS1411-TP | | | TP | | | AS1411 | | |
|---|---|---|---|---|---|---|---|---|---|
| (nM) | sample 1 | sample 2 | sample 3 | sample 1 | sample 2 | sample 3 | sample 1 | sample 2 | sample 3 |
| 200.0 | 81.6194 | 83.0397 | 85.1327 | 93.9952 | 99.8060 | 96.6312 | 8.3084 | 8.0987 | 13.5426 |
| 100.0 | 70.1522 | 69.0930 | 72.8414 | 96.2778 | 97.7552 | 82.0308 | 7.6842 | 6.8369 | 7.5344 |
| 50.0 | 54.6914 | 44.4723 | 45.0792 | 88.6995 | 87.4474 | 84.6990 | 7.3070 | 6.4174 | 7.0396 |
| 25.0 | 32.9996 | 30.9061 | 32.0759 | 74.1333 | 73.3084 | 73.3954 | 4.5962 | 5.2695 | 5.1506 |
| 12.5 | 20.2720 | 15.9757 | 14.3037 | 66.3653 | 59.2076 | 65.5783 | 3.1924 | 2.7446 | 2.0042 |
| 6.3 | 7.0403 | 6.2799 | 10.7730 | 31.9827 | 40.8401 | 42.6158 | 2.1198 | 1.5844 | 2.2503 |
| 3.1 | 5.5086 | 6.0304 | 3.7088 | 21.4060 | 23.3551 | 17.9111 | 0.4510 | 1.1866 | 0.8691 |

This can be seen by combining the experimental data in Tables 6-7 above and the statistical line graphs of the inhibitory effects of the drugs on cell proliferation in normal hepatocytes LO2 and normal human embryonic kidney cells HEK293 shown in FIGS. 8-9 that,

Triptolide has a large cytotoxicity against normal hepatocytes LO2 and normal human embryonic kidney cells HEK293, with IC₅₀ of 9.01±0.25 nM and 5.98±0.21 nM, respectively, and its cytotoxicity was reduced when triptolide conjugated with the aptamer AS1411, and the IC₅₀s of the conjugate against LO2 and HEK293 were 51.86±0.13 nM and 48.10±0.13 nM, respectively, and AS1411 had no significant inhibitory effect on both normal cells. This fully proved that the aptamer attachment reduced the cytotoxicity of triptolide to normal cells.

The reasons may exist in the following two aspects: 1. The aptamer closes the active group of triptolide, normal cells are non-weakly acidic environment, triptolide cannot be released intact. 2. Normal hepatocytes and renal cells have less expression of nucleolin on the surface of the cell membrane, the AS1411 aptamer does not have targeting to the normal cells, and the cellular uptake of conjugate is less, and the toxic effect is small.

### IV. In Vivo Distribution Study of AS1411-TP

In order to verify the in vivo distribution of AS1411-TP conjugate, in vivo distribution experiments were performed on mouse models of colon cancer, breast cancer and lung cancer, respectively, and the content of the conjugate in their tissues was determined by HPLC to study the in vivo distribution pattern, and the following are the in vivo distribution studies performed in different tumor-bearing mice, respectively.

### 4.1. Colon Cancer Cell Line HCT116

Materials: the dose of AS1411-TP administration group was set at 50 μM, and the volume of administration was 100 μL; the dose of triptolide (TP) administration group was set at 50 μM, and the volume of administration was 100 L. The same materials as those described in Part II above will not be repeated here. Additionally, the experimental animals: BALB/cnu mice, female, 6-7 weeks age, provided by SPF (Beijing) Biotechnology Co. Ltd.; housed in the laboratory animal room of Chengdu University of Traditional Chinese Medicine.

Experimental protocol: female nude mice were reared for 6-7 weeks under standard environment, colon cancer cells HCT116 were suspended in serum-free medium, and each mouse was inoculated with 3*10⁶ tumor cells. When the tumor volume reached 50-100 mm³, the mice were randomly divided into two groups and injected with AS1411-TP and TP by tail vein, 12 animals in each group. After 8 h of drug administration, the animals were executed, and the heart, liver, spleen, lung, kidney and tumor tissues were removed, rinsed with saline, weighed, and frozen at −80° C. for a short period of time. Take the tissues of each organ, add 2 times the amount of saline, homogenize and centrifugal filtration, take 90 μL of filtrate, add 10 μL of standard solution into the filtrate, and then determine the drug content of the tissues by HPLC after different time of drug administration. The distribution of the above drugs in different tissues of the colon cancer model of loaded mice is shown in FIG. 17.

The distribution results in FIG. 17 showed that AS1411-TP was more distributed in colon cancer tumor tissues and less distributed in normal tissues, and TP was more distributed in normal liver and kidney tissue tissues. The amount of AS1411-TP in normal tissues of mice was much lower than that of TP, while in tumor tissues, compared with the triptolide experimental group, the amount of AS1411-TP in the tumor tissue accounts for a higher percentage of the total injection, which indicates that AS1411-TP has a good targeting effect on colon cancer tumor tissue in vivo.

### 4.2. Breast Cancer Cell Line MDA-MB-231

The materials in this procedure that are the same as those in 4.1 are not repeated here, only the cancer cell lines were replaced.

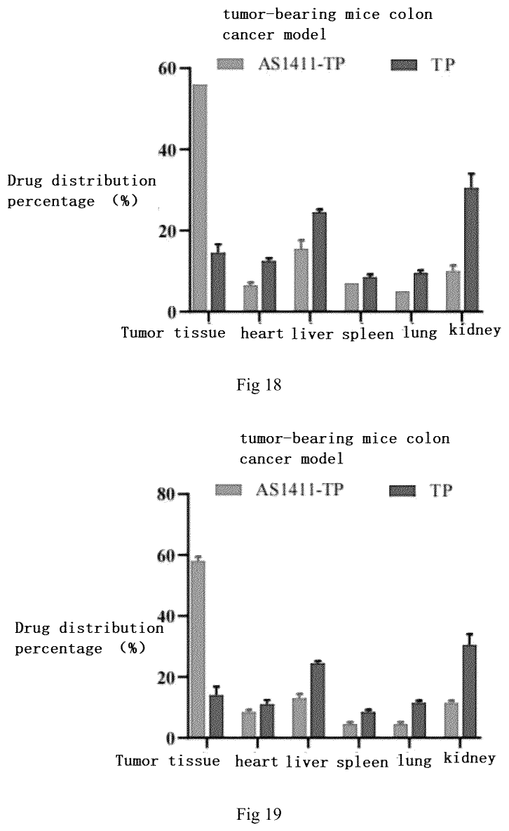

Experimental protocol: female nude mice were reared for 6-7 weeks under standard environment, breast cancer cells MDA-MB-231 were suspended in serum-free medium, and each mouse was inoculated with 7*10⁶ tumor cells. When the tumor volume reached 50-100 mm³, the mice were randomly divided into two groups and injected with the adapter—Triptolide conjugate and Triptolide by tail vein, 12 animals in each group. After 4 h of drug administration, the animals were executed, and the heart, liver, spleen, lung, kidney and tumor tissues were removed, rinsed with saline, weighed, and frozen at −80° C. for a short period of time. Detection of the content of aptamer—Triptolide and Triptolide in tissues by HPLC. Take the tissues of each organ, add 2 times the amount of saline, homogenize and centrifugal filtration, take 90 μL of filtrate, add 10 μL of standard solution into the filtrate, and then determine the drug content of the tissues by HPLC after different time of drug administration. The distribution of the above drugs in different tissues of the colon cancer model of loaded mice is shown in FIG. 18.

The distribution results in FIG. 18 showed that AS1411-TP was more distributed in colon cancer tumor tissues and less distributed in normal tissues, and triptolide was more distributed in normal liver and kidney tissue tissues. The amount of AS1411-TP in normal tissues of mice was much lower than that of triptolide, while in tumor tissues, compared with the triptolide experimental group, the amount of AS1411-TP in the tumor tissue accounts for a higher percentage of the total injection, which indicates that AS1411-TP has a good targeting effect on colon cancer tumor tissue in vivo.

### 4.3. Lung Cancer Cell Line A549

The materials in this procedure that are the same as those in 4.2 are not repeated here, only the cancer cell lines were replaced.

Experimental protocol: female nude mice were reared for 6-7 weeks under standard environment, lung cancer cells A549 were suspended in serum-free medium, and each mouse was inoculated with 6*10⁶ tumor cells. When the tumor volume reached 50-100 mm³, the mice were randomly divided into two groups and injected with the adapter—Triptolide conjugate and Triptolide by tail vein, 12 animals in each group. After 4 h of drug administration, the animals were executed, and the heart, liver, spleen, lung, kidney and tumor tissues were removed, rinsed with saline, weighed, and frozen at −20° C. for a short period of time. Detection of the content of AS1411-TP and TP in tissues by HPLC. Detection of the content of aptamer—Triptolide and Triptolide in tissues by HPLC. Take the tissues of each organ, add 2 times the amount of saline, homogenize and centrifugal filtration, take 90 μL of filtrate, add 10 μL of standard solution into the filtrate, and then determine the drug content of the tissues by HPLC after different time of drug administration. The distribution of the above drugs in different tissues of the colon cancer model of loaded mice is shown in FIG. 19.

The distribution results in FIG. 19 showed that AS1411-TP was more distributed in colon cancer tumor tissues and less distributed in normal tissues, and triptolide was more distributed in normal liver and kidney tissue tissues. The amount of AS1411-TP in normal tissues of mice was much lower than that of triptolide, while in tumor tissues, compared with the triptolide experimental group, the amount of AS1411-TP in the tumor tissue accounts for a higher percentage of the total injection, which indicates that AS1411-TP has better tumor targeting of lung cancer cell A549 in vivo, while circumventing other tissues to achieve the effect of tumor targeting.

### V. Experimental Studies on the In Vivo Activity of AS1411-TP

In order to verify the in vivo antitumor effects of AS1411-TP conjugate, in vivo antitumor experiments were conducted on colon, breast and lung cancer mouse models, respectively, and the tumor weight, RTV value, tumor inhibition rate and T/C value were measured to verify the antitumor effects. The following are the in vivo activity studies conducted in different tumor-bearing mice:

### 5.1. Colon Cancer Cell Line HCT116

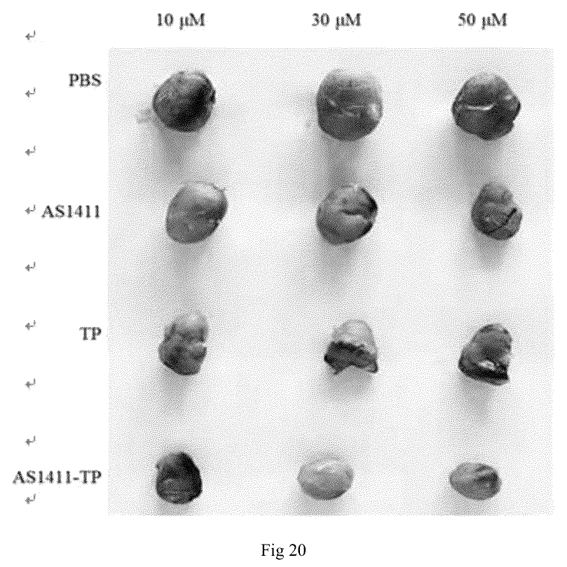

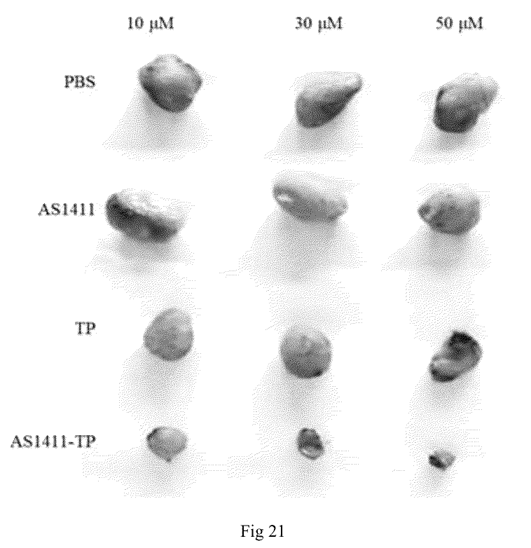

Female nude mice were reared for 6-7 weeks under standard environment, colon cancer cells HCT116 were suspended in serum-free medium, and each mouse was inoculated with 5*10⁶ tumor cells. When the tumor volume reached 50-100 mm³, the mice were randomly divided into 4 groups, and were injected with aptamer triptolide conjugate, triptolide, and aptamer by tail vein, respectively, 12 mice in each group. A PBS negative control group was set up. Administration groups: TP (10 μM, 30 μM and 50 μM, administered in a volume of 100 μL), AS1411 (10 μM, 30 μM and 50 μM, administered in a volume of 100 μL) and AS1411-TP (10 μM, 30 μM and 50 μM, administered in a volume of 100 μL). The drugs were administered once every 2 days for 8 consecutive doses, and the experiment was ended 24 hours after the last dose. At the end of the experiment, the animals were executed by breaking the cervical vertebrae, and the tumors were stripped and weighed to calculate the tumor growth inhibition rate of the drug. The t-test was used to compare the tumor weight, tumor volume, RTV and other indexes of the animals in each group. The experimental data were summarized in Table 8, and FIG. 20 demonstrated the anti-mouse colon cancer xenograft tumors obtained from different injected drugs.

Table 8 shows a summary of the experimental data on the growth inhibitory effects of AS1411-TP, TP, and AS1411 on the tumor-bearing mouse colon cancer tumor model:

| | | Tumor | | |
|---|---|---|---|---|
| | Tumor | inhibition | | T/C |
| Group | weight | rate (%) | RTV | (%) |
| PBS | 3.11 ± 0.21 | | 35.55 ± 0.31 | |
| AS1411(10 μM) | 2.92 ± 0.33 | 6.45 | 33.13 ± 0.51 | 93.19 |
| AS1411(30 μM) | 2.74 ± 0.15 | 12.91 | 28.56 ± 0.23 | 80.34 |
| AS1411(50 μM) | 2.66 ± 0.14 | 16.12 | 26.32 ± 0.15 | 74.04 |
| AS1411-TP(10 μM) | 2.21 ± 0.35 | 29.03 | 21.48 ± 0.45 | 60.42 |
| AS1411-TP(30 μM) | 1.54 ± 0.37 | 51.61 | 15.76 ± 0.15 | 44.33 |
| AS1411-TP(50 μM) | 0.97 ± 0.11 | 70.96 | 8.34 ± 0.22 | 23.46 |
| TP(10 μM) | 2.31 ± 0.24 | 25.80 | 23.31 ± 0.31 | 65.57 |
| TP(30 μM) | 1.76 ± 0.13 | 45.16 | 16.68 ± 0.19 | 46.92 |
| TP(50 μM) | 1.11 ± 0.25 | 64.51 | 10.09 ± 0.28 | 28.38 |

During the experimental observation, the body weights of mice in TP, AS1411-TP and AS1411 groups were basically maintained in the tolerable range of animal toxicity and side-effects during 8 times of administration. The body weights of mice in the PBS-negative control group showed a slow and gradual decreasing trend, with an average body weight reduction of 1.7 g compared with that at the beginning of the grouping. The weights of the tumors of the animals, the tumor inhibitionrate, and the RTV values showed that the tumor growth rate and tumor inhibition rate of mice in the TP group, AS1411-TP group and AS1411 group showed a dose-dependent relationship compared with that of the PBS group. Among them, the administration of AS1411-TP and TP groups showed a significant dose-response relationship on tumor growth rate and tumor inhibition rate in animals. However, all the indicators showed that the tumor inhibitory effect of the TP group was lower than that of the AS1411-TP group at the corresponding concentration.

Conclusion: AS1411-TP 10 μM, 30 μM and 50 μM were administered to the tail of nude mice, a model of colon cancer, for 8 consecutive intravenous injections, and tumor growth was significantly inhibited, and the inhibition was better than that of TP administered at the same concentration, indicating that AS1411-TP has a better anti-tumor effect in vivo.

### 5.2. Breast Cancer Cell Line MDA-MB-231

The materials used in the experiments were the same as those tested in 5.1 Colon cancer cell lines, differing only in the tumor strains.

Specific experimental protocol: Female nude mice were reared for 6-7 weeks under standard environment, breast cancer cells MDA-MB-231 were suspended in serum-free medium, and each mouse was inoculated with 6*10⁶ tumor cells. When the tumor volume reached 50-100 mm³, the mice were randomly divided into 4 groups, and were injected with aptamer triptolide conjugate, triptolide, and aptamer by tail vein, respectively, 12 mice in each group. A PBS negative control group was set up. Administration groups: triptolide (10 μM, 30 μM and 50 μM), AS1411 (10 μM, 30 μM and 50 μM) and AS1411-TP (10 μM, 30 μM and 50 μM). The drugs were administered once every 2 days for 8 consecutive doses, and the experiment was ended 24 hours after the last dose. At the end of the experiment, the animals were executed by breaking the cervical vertebrae, and the tumors were stripped and weighed to calculate the tumor growth inhibition rate of the drug. The t-test was used to compare the tumor weight, tumor volume, RTV and other indexes of the animals in each group. The experimental data were summarized in Table 9, and FIG. 21 demonstrated the anti-mouse breast cancer xenograft tumors obtained from different injected drugs.

Table 9 shows a summary of the experimental data on the growth inhibitory effects of AS1411-TP, TP, and AS1411 on the tumor-bearing mouse breast cancer tumor model:

| | | Tumor | | |
|---|---|---|---|---|
| | Tumor | inhibition | | T/C |
| Group | weight | rate (%) | RTV | (%) |
| PBS | 3.43 ± 0.27 | | 41.45 ± 0.12 | |
| AS1411(10 μM) | 3.19 ± 0.27 | 7.00 | 39.29 ± 16 | 94.79 |
| AS1411(30 μM) | 2.92 ± 0.39 | 14.87 | 37.55 ± 34 | 90.59 |
| AS1411(50 μM) | 2.78 ± 0.41 | 18.95 | 35.19 ± 38 | 84.90 |
| AS1411-TP(10 μM) | 2.13 ± 0.17 | 37.90 | 24.53 ± 45 | 59.18 |
| AS1411-TP(30 μM) | 1.41 ± 0.11 | 58.89 | 13.56 ± 14 | 32.71 |
| AS1411-TP(50 μM) | 0.88 ± 0.26 | 74.34 | 9.77 ± 22 | 23.57 |
| TP(10 μM) | 2.34 ± 0.39 | 31.78 | 27.18 ± 47 | 65.57 |
| TP(30 μM) | 1.77 ± 0.51 | 48.40 | 16.44 ± 31 | 39.66 |
| TP(50 μM) | 0.89 ± 0.08 | 74.05 | 12.89 ± 55 | 31.10 |

During the experimental observation, the body weights of mice in TP, AS1411-TP and AS1411 groups were basically maintained in the tolerable range of animal toxicity and side-effects during 8 times of administration. The body weights of mice in the PBS-negative control group showed a slow and gradual decreasing trend, with an average body weight reduction of 2.1 g compared with that at the beginning of the grouping. The weights of the tumors of the animals, the tumor inhibition rate, and the RTV values showed that the tumor growth rate and tumor inhibition rate of mice in the TP group, AS1411-TP group and AS1411 group showed a dose-dependent relationship compared with that of the PBS group. Among them, the administration of AS1411-TP and TP groups showed a significant dose-response relationship on tumor growth rate and tumor inhibition rate in animals. However, all the indicators showed that the tumor inhibitory effect of the TP group was lower than that of the AS1411-TP group at the corresponding concentration.

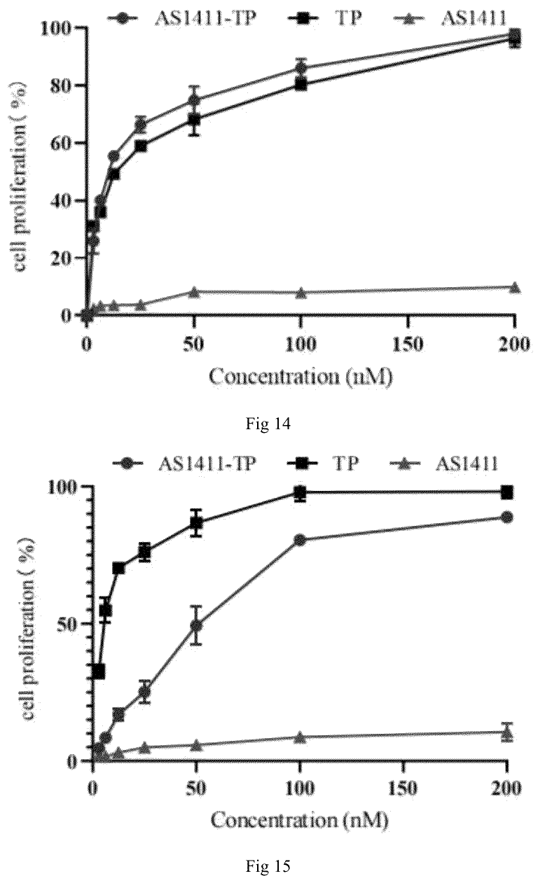

Through the corresponding display results in Table 9 and FIG. 14, it can be concluded that AS1411-TP 10 μM, 30 μM and 50 μM administered by tail vein injection for eight consecutive times to the nude mice model of breast cancer, the growth of breast cancer tumor cells was significantly inhibited, the inhibition efficiency was significantly correlated with the dose of the drug administered, and the inhibition was better than that of the drug administered at the same concentration of TP, which indicated that AS1411-TP had a better in vivo effect of AS1411-TP on breast cancer.

### 5.3. Lung Cancer Cell Line A549

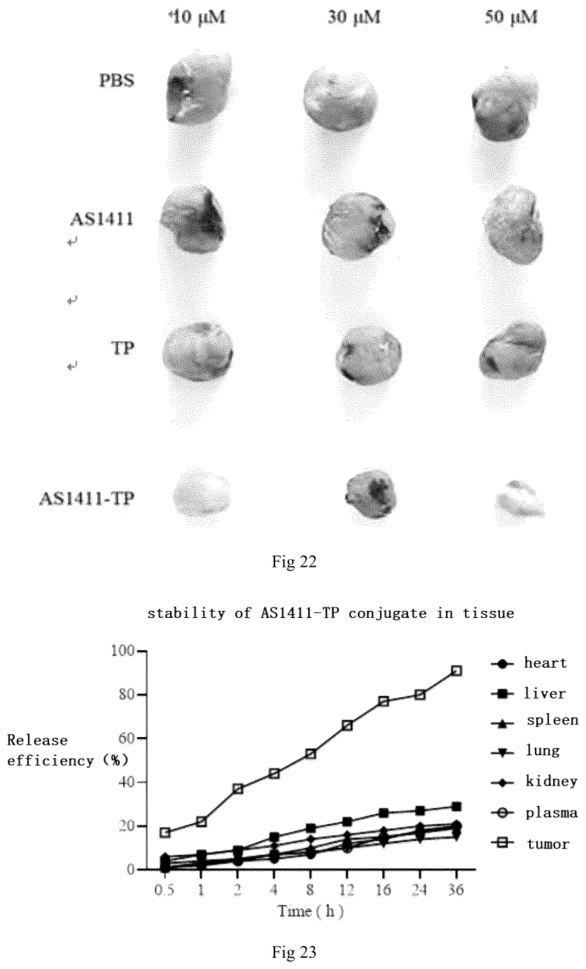

Experimental protocol: Female nude mice were reared for 6-7 weeks under standard environment, lung cancer cell line A549 were suspended in serum-free medium, and each mouse was inoculated with 6*10⁶ tumor cells. When the tumor volume reached 50-100 mm³, the mice were randomly divided into 4 groups, and were injected with aptamer triptolide conjugate, triptolide, and aptamer by tail vein, respectively, 12 mice in each group. A PBS negative control group was set up. Administration groups: TP (10 μM, 30 μM, and 50 μM), AS1411 (10 μM, 30 μM, and 50 μM), and AS1411-TP (10 μM, 30 μM, and 50 μM). The drugs were administered once every 2 days for 8 consecutive doses, and the experiment was ended 24 hours after the last dose. At the end of the experiment, the animals were executed by breaking the cervical vertebrae, and the tumors were stripped and weighed to calculate the tumor growth inhibition rate of the drug. The t-test was used to compare the tumor weight, tumor volume, RTV and other indexes of the animals in each group. The experimental data were summarized in Table 10, and FIG. 22 demonstrated the anti-mouse breast cancer xenograft tumors obtained from different injected drugs.

Table 9 shows a summary of the experimental data on the growth inhibitory effects of AS1411-TP, TP, and AS1411 on the tumor-bearing mouse lung cancer tumor model.

| | | Tumor | | |
|---|---|---|---|---|
| | Tumor | inhibition | | T/C |
| Group | weight | rate (%) | RTV | (%) |
| PBS | 3.33 ± 0.34 | | 37.13 ± 0.11 | |
| AS1411 (10 μM) | 3.16 ± 0.13 | 6.06 | 34.57 ± 0.18 | 89.93 |
| AS1411 (3 0 μM) | 2.88 ± 0.26 | 15.15 | 31.09 ± 0.27 | 75.76 |
| AS1411 (50 μM) | 2.61 ± 0.38 | 21.21 | 26.19 ± 0.38 | 70.06 |
| AS1411-TP (10 μM) | 2.29 ± 0.27 | 33.33 | 24.22 ± 0.55 | 40.79 |
| AS1411-TP (30 μM) | 1.51 ± 0.19 | 54.55 | 14.1 ± 0.26 | 26.55 |
| AS1411-TP (50 μM) | 1.15 ± 0.16 | 66.67 | 9.18 ± 0.37 | 75.50 |
| TP (10 μM) | 2.47 ± 0.08 | 27.27 | 26.1 ± 0.09 | 51.81 |
| TP (30 μM) | 1.79 ± 0.19 | 48.48 | 17.91 ± 0.27 | 31.59 |
| TP (50 μM) | 1.29 ± 0.27 | 63.64 | 10.92 ± 0.15 | 89.93 |

During the experimental observation, the body weights of mice in TP, AS1411-TP and AS1411 groups were basically maintained in the tolerable range of animal toxicity and side-effects during 8 times of administration. The body weights of mice in the PBS-negative control group showed a slow and gradual decreasing trend, with an average body weight reduction of 2.0 g compared with that at the beginning of the grouping. The weights of the tumors of the animals, the tumor inhibition rate, and the RTV values showed that the tumor growth rate and tumor inhibition rate of mice in the TP group, AS1411-TP group and AS1411 group showed a dose-dependent relationship compared with that of the PBS group. Among them, the administration of AS1411-TP and TP groups showed a significant dose-response relationship on tumor growth rate and tumor inhibition rate in animals. However, all the indicators showed that the tumor inhibitory effect of the TP group was lower than that of the AS1411-TP group at the corresponding concentration.

Through the corresponding display results in Table 10 and FIG. 22, it can be concluded that AS1411-TP 10 μM, 30 μM and 50 μM were administered to lung adenocarcinoma cell line A549 nude mice by tail vein injection for 8 consecutive times, and the tumor growth of the lung cancer cell line A549 was significantly inhibited, and the inhibition efficiency was significantly correlated with the administered dose, and better than that administered at the same concentration of TP, indicating that AS1411-TP has a good targeting inhibition effect on lung adenocarcinoma, indicating that AS1411-TP has a better targeting inhibitory effect on lung cancer.

### VI. In Vivo Stability Studies of AS1411-TP

In vivo stability experiments in mice were performed on the aptamer triptolide conjugate to mimic the stability of the drug in humans in order to verify the dissociation and stability of the conjugate in different tissues.

Experimental methods: 27 tumor-bearing mice were randomly selected and injected with 100 μL of saline solution at a concentration of 50 μM AS1411-TP into the tailbone vein. 3 mice were executed at 0.5, 1, 2, 4, 8, 12, 16, 24, and 32 h, respectively, and anesthetized by intraperitoneal injection of sodium pentobarbital, and the tissues of heart, liver, spleen, lungs, kidneys, and solid tumors were taken by cardiac puncture to obtain blood. Tissue homogenization, extraction, sample processing and pre-experimentation were carried out to determine the retention time of TP, AS1411 and AS1411-TP conjugate, respectively, and then HPLC detected the amount of free TP and AS1411-TP in each tissue at each time point, respectively, and the release efficiencies were calculated, to monitor and quantitatively examine the stability of the drug in the tissues. The experimental data are shown in Table 11, and the results of the in vivo stability test data analysis are shown in FIG. 23.

Table 11 shows the in vivo stability test data for the aptamer triptolide conjugate (AS1411-TP):

| | Release rate of drugs in each tissue at each time point | | | | | | | | |
|---|---|---|---|---|---|---|---|---|---|
| | ½ h | 1 h | 2 h | 4 h | 8 h | 12 h | 16 h | 24 h | 36 h |
| heart | 1% | 2% | 4% | 5% | 7% | 12% | 14% | 18% | 20% |
| liver | 4% | 7% | 9% | 15% | 19% | 22% | 26% | 27% | 29% |
| spleen | 1% | 3% | 5% | 7% | 10% | 14% | 15% | 17% | 19% |
| lung | 3% | 4% | 5% | 7% | 8% | 10% | 12% | 14% | 15% |
| kidney | 6% | 7% | 9% | 11% | 14% | 16% | 18% | 20% | 21% |
| plasma | 1% | 3% | 4% | 7% | 8% | 10% | 15% | 17% | 20% |
| tumor | 17% | 22% | 37% | 44% | 53% | 66% | 77% | 80% | 91% |

As shown in FIG. 23 which is the data analysis graph of in vivo stability test of aptamer triptolide conjugate (AS1411-TP), the data in Table 11 and FIG. 16 can show that: AS1411-TP conjugate is almost not dissociated or only a small amount of dissociation in plasma and normal tissues, such as heart, spleen, and lungs, after 36 hours, and a little more dissociation is observed in renal and liver tissues, and in tumor tissues, the AS1411-TP conjugate was dissociated in large amounts and the degree of dissociation increased with the accumulation of time. The above results demonstrated that the linking bond used to connect triptolide and the aptamer played a significant role in vivo, such that the conjugate does not cleave in normal tissues and has less toxicity; while it cleaves in tumor tissues, releasing triptolide and exerting therapeutic effects.

Conclusion: After synthesizing the above experimental studies on various aspects of the drug, the present invention provides an aptamer triptolide conjugate with an acetal ester functional group as the linking bond. This linking bond has a better weak acid responsiveness with a chain cleaving pH of 3.5-6.5 compared to the existing enol ether bond, whereas the enol ether bond is cleaved at pH=3.5 or less, so for the tumor microenvironment, the aptamer triptolide conjugate provided by the present invention is more responsive to the tumor microenvironment. Meanwhile, in vitro experiments proved that the aptamer triptolide conjugate provided by the present invention has better serum stability.

The aptamer triptolide conjugate provided by the present invention has comparable anti-tumor effect with the original drug both in vivo and in vitro, which proves that the connection of the aptamer does not affect the anti-tumor effect of the conjugate. At the same time, both in vivo and in vitro experiments showed that the toxicity of the conjugate was reduced compared with that of the original drug, which proved that the conjugate had a better targeted anti-tumor effect, and at the same time, the toxicity to normal tissues could be circumvented.

The distribution of the aptamer triptolide conjugate provided by the present invention in tumor tissues is much higher than that in other tissues, and the dissociation efficiency in tumor tissues is much higher than that in normal tissues at the same time, which proves that the conjugate provided by the present invention has a better tumor-targeting property in vivo, and at the same time, the tumor microenvironment can satisfy the conditions for the release of the conjugate.

The foregoing are only preferred embodiments of the present invention, and it should be understood that the present invention is not limited to the forms disclosed herein, and should not be regarded as exclusive of other embodiments, but may be utilized in a variety of other combinations, modifications, and environments, and is capable of being altered by the above teachings, or by skill or knowledge in the relevant fields, within the scope of the concepts hereinbefore described. And the alterations and variations made by those in the art that do not depart from the spirit and scope of the present invention shall all be within the scope of protection of the appended claims of the present invention.

1. An acid-sensitive aptamer triptolide conjugate, characterized in having the following general formula: 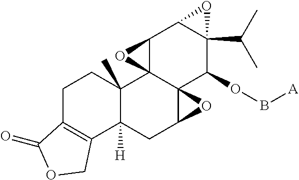
wherein, A is a nucleic acid aptamer;
B is a linking bond linking triptolide to the nucleic acid aptamer, and said linking bond has an acetal ester functional group formed at the position 14 hydroxyl of triptolide.

2. An acid-sensitive aptamer triptolide conjugate, characterized in having the following general formula: 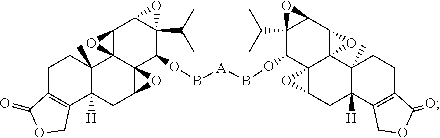
wherein A is a nucleic acid aptamer;
B is a linking bond linking triptolide to the nucleic acid aptamer, and said linking bond has an acetal ester functional group formed at the position 14 hydroxyl of triptolide.

3. The acid-sensitive aptamer triptolide conjugate according to claim 1, wherein the linking bond B has a structure of the following general formula:

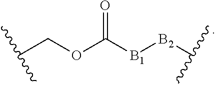

4. The acid-sensitive aptamer triptolide conjugate according to claim 3, wherein the structure of B₁ has the following general structure: 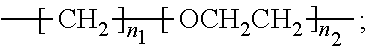
wherein 0≤n₁≤100, preferably, 0≤n₁≤50, more preferably, 0≤n₁≤20;
wherein 0≤n₂≤100, preferably, 0≤n₂≤50, more preferably, 0≤n₂≤20;
n₁ and n₂ take values in the range including 0, with the provision that they are not both 0;
specifically, n₁, n₂ preferably take values of 2, 3, 5, 10.

5. The acid-sensitive aptamer triptolide conjugate according to claim 3, wherein B₂ has the following general formula:

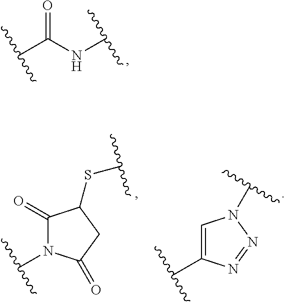

6. The acid-sensitive aptamer triptolide conjugate according to claim 1, wherein said nucleic acid aptamer is a modified nucleic acid aptamer, and said modified nucleic acid aptamer has the general structure of A₁-C₁, wherein the structure of C₁ is: 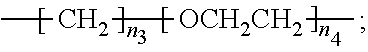
wherein 0≤n₃≤100, preferably, 0≤n₃≤50, more preferably, 0≤n₃≤20;
wherein 0≤n₄≤100, preferably, 0≤n₄≤50, more preferably, 0≤n₄≤20;
n₃ and n₄ take values in the range including 0, with the provision that they are not both 0;
specifically, n₃, n₄ preferably take values of 2, 3, 5, 10.

7. The acid-sensitive aptamer triptolide conjugate according to claim 6, wherein A1 is any one of AS1411, Pegaptanib, Sgc8c, A10, DNA aptamer, RNA aptamer, CL4, Apt and E07.

8. The acid-sensitive aptamer triptolide conjugate according to claim 7, wherein the sequence of A1 is shown below:
AS1411:
(SEQ ID NO: 1)
GGTGGTGGTGGTTGTGGTGGTGGTGG;
Pegaptanib:
(SEQ ID NO: 2)
GCGAACCGAUGGAAUUUUUGGACGCUCGC;
Sgc8c:
(SEQ ID NO: 3)
ATCTAACTGCTGCGCCGCCGGGAAAATACTGTACGGTTAGA;
A10:
(SEQ ID NO: 4)
GGGAGGACGAUGCGGAUCAGCCAUGUUUACGUCACUCCUUGUCAAUCCUC
AUCGGCAGACGACUCGCCCGA;
DNA aptamer:
(SEQ ID NO: 5)
GGGAGACAAGAATAAACGCTCAA-(25N)-
TTCGACAGGAGGCTCACAACAGGC;
RNA aptamer:
(SEQ ID NO: 6)
GGGGGCAUACUUGUGAGACUUUUAUGUCACCCCC;
CL4:
(SEQ ID NO: 7)
GCCUUAGUAACGUGCUUUGAUGUCGAUUCGACAGGAGGC;
Apt:
(SEQ ID NO: 8)
GCAGTTGATCCTTTGGATACCCTGG;
E07:
(SEQ ID NO: 9)
GGACGGAUUUAAUCGCCGUAGAAAAGCAUGUCAAAGCCGGAACCGUCC.

9. A method of preparing the acid-sensitive aptamer triptolide conjugate according to claim 8, comprising the following steps to obtain the same:
Step 1: Preparation of a triptolide derivative, wherein the position 14 hydroxyl of triptolide is connected to an acetal ester functional group;
Step 2: Modification of a nucleic acid aptamer to obtain a modified nucleic acid aptamer modifier A₁-C₁;
Step 3: Linking the triptolide derivative with the nucleic acid aptamer modifier in Step 2 via a B₂ linker, to obtain the acid-sensitive aptamer triptolide conjugate.

10. Use of the acid-sensitive triptolide aptamer conjugate according to claim 1 in the preparation of an antitumor drug.

11. The acid-sensitive aptamer triptolide conjugate according to claim 2, wherein the linking bond B has a structure of the following general formula:

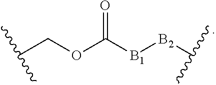

12. The acid-sensitive aptamer triptolide conjugate according to claim 11, wherein the structure of B₁ has the following general structure: 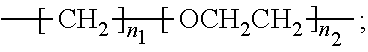
wherein 0≤n₁≤100, preferably, 0≤n₁≤50, more preferably, 0≤n₁≤20;
wherein 0≤n₂≤100, preferably, 0≤n₂≤50, more preferably, 0≤n₂≤20;
n₁ and n₂ take values in the range including 0, with the provision that they are not both 0;
specifically, n₁, n₂ preferably take values of 2, 3, 5, 10.

13. The acid-sensitive aptamer triptolide conjugate according to claim 11, wherein B₂ has the following general formula:

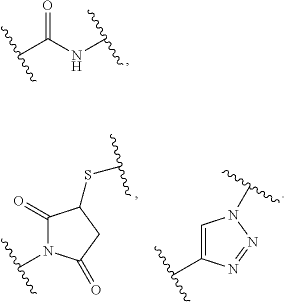

14. Use of the acid-sensitive triptolide aptamer conjugate according to claim 2 in the preparation of an antitumor drug.


SEQUENCE LISTING (9 sequences)

SEQ ID NO: 1 - DNA, 26 bp, synthetic construct
  source 1..26: mol_type = other DNA
ggtggtggtggttgtggtggtggtgg

SEQ ID NO: 2 - RNA, 29 nt, synthetic construct
  source 1..29: mol_type = other RNA
gcgaaccgatggaatttttggacgctcgc

SEQ ID NO: 3 - DNA, 41 bp, synthetic construct
  source 1..41: mol_type = other DNA
atctaactgctgcgccgccgggaaaatactgtacggttaga

SEQ ID NO: 4 - RNA, 71 nt, synthetic construct
  source 1..71: mol_type = other RNA
gggaggacgatgcggatcagccatgtttacgtcactccttgtcaatcctcatcggcagacgactcgcccga

SEQ ID NO: 5 - DNA, 72 bp, synthetic construct
  source 1..72: mol_type = other DNA
gggagacaagaataaacgctcaannnnnnnnnnnnnnnnnnnnnnnnnttcgacaggaggctcacaacaggc

SEQ ID NO: 6 - RNA, 34 nt, synthetic construct
  source 1..34: mol_type = other RNA
gggggcatacttgtgagacttttatgtcaccccc

SEQ ID NO: 7 - RNA, 39 nt, synthetic construct
  source 1..39: mol_type = other RNA
gccttagtaacgtgctttgatgtcgattcgacaggaggc

SEQ ID NO: 8 - DNA, 25 bp, synthetic construct
  source 1..25: mol_type = other DNA
gcagttgatcctttggataccctgg

SEQ ID NO: 9 - RNA, 48 nt, synthetic construct
  source 1..48: mol_type = other RNA
ggacggatttaatcgccgtagaaaagcatgtcaaagccggaaccgtcc